SEQUENCE LISTING (16 sequences)

SEQ ID NO: 1 - DNA, 12012 bp, synthetic construct
  source 1..12012: mol_type = other DNA
ccaccgcctcggacctggccgacctcatcttccccgagcgcctcaggctgacgctgcgcaacggcctgcccgactttatgagccaaagcatgttgcaaaaactttcggtaaaaaatgtaattactccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatataagccctaacctacactgacgtaaaagggcccaaaagtctaaaaaaataacccgcccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatggtttaaacgcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagacggaaaaacatatcagaacgttcagcagtttatcgatgaaggcaattatacatccggcgacaaccatacgctgagagaccctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcaaactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttgcgccaagcttcttaatgaacatcaaaggcaataaaacatccgttgtcaaaaacagcatcctggagcaaggacagctgacagtcaactaataacagcaaaaagaaaatgccgatacttcattggcattttcttttatttctcaacaagatggtgaattgactagtgggtagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattgatccggaacccttaatataacttcgtataatgtatgctatacgaagttattaggtccctcgactatagggtcaccgtcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacrayacgagtgggaycgtggtcccagactaataatcagaccgacgatacgagtgggaccgtggtcccagactaataatcagaccgacgatacgagtgggaccgtggtyccagwctratwatcagaccgacgatacragtggracmgtggkcccagasakaatawtcagrccgagwtaygcwktckggcctgtaacaaaggacattaagtaaagacagatamrmgtgrgactaaaacgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcaactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacggcgcgccaaagcttgcatgcctgcagccgcgtaacctggcaaaatcggttacggttgagtaataaatggatgccctgcgtaagcggggcacatttcattacctctttctccgcacccgacatagataataacttcgtatagtatacattatacgaagttatctagtagacttaatcgcgtttaaacccatcatcaataatatacctcaaactttttgtgcgcgttaatatgcaaatgaggcgtttgaatttgggaagggaggaaggtgattggccgagagaagggcgaccgttaggggcggggcgagtgacgttttgatgacgtgaccgcgaggaggagccagtttgcaagttctcgtgggaaaagtgacgtcaaacgaggtgtggtttgaacacggaaatactcaattttcccgcgctctctgacaggaaatgaggtgtttctaggcggatgcaagtgaaaacgggccattttcgcgcgaaaactgaatgaggaagtgaaaatctgagtaatttcgcgtttatgacagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgcgcgagtcagatctacactttgaaaggcgatcgctagcgacatcgatcacaagtttgtacaaaaaagcaggctccaccatgggaacccgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtacc

SEQ ID NO: 2 - DNA, 29187 bp, synthetic construct
  source 1..29187: mol_type = other DNA
  misc_feature 1..29187: note = Vector
gcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatcccgtcgagatatctagacccagctttcttgtacaaagtggtgatcgattcgacagatcgcgatcgcagtgagtagtgttctggggcgggggaggacctgcatgagggccagaatgactgaaatctgtgcttttctgtgtgttgcagcatcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcggtggacgcagctgccgccgcagctgctgcatccgccgccagcgccgtgcgcggaatggccatgggcgccggctactacggcactctggtggccaactcgagttccaccaataatcccgccagcctgaacgaggagaagctgctgctgctgatggcccagcttgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgtccgcccaggttttccatgcactcatccatgatgatggcaatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcagagggggggatcatgtccacctgcggggcgataaagaacacggtttccggggcgggggagatgagctgggccgaaagcaagttccggagcagctgggacttgccgcagccggtggggccgtaaatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagagataggagctcctggagcgaggcgaagtttttcagcggcttgagtccgtcggccatgggcattttggagagggtctgttgcaagagttccaagcggtcccagagctcggtgatgtgctctacggcatctcgatccagcagacctcctcgtttcgcgggttgggacgactgcgggagtagggcaccagacgatgggcgtccagcgcagccagggtccggtccttccagggccgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggaatcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccactttttccagggtatgcaaacacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccagcggagatgcctttcaagagcccctcgtccatctggtcagaaaagacgatttttttgttgtcgagcttggtggcgaaggagccgtagagggcgttggaaaggagcttggcgatggagcgcatggtctggtttttttccttgtcggcgcgctccttggccgcgatgttgagctgcacgtactcgcgcgccacgcacttccattcggggaagacggtggtcatctcgtcgggcacgattctgacctgccaacctcgattatgcagggtgatgaggtccacactggtggccacctcgccgcgcaggggctcgttggtccagcagaggcggccgcccttgcgcgagcagaaggggggcagagggtccagcatgacctcgtcgggggggtcggcatcgatggtgaagatgccgggcaggagatcggggtcgaagtagctgatggaagtggccagatcgtccagggaagcttgccattcgcgcacggccagcgcgcgctcgtagggactgaggggcgtgccccagggcatggggtgggtgagcgcggaggcgtacatgccgcagatgtcgtagacgtagaggggctcctcgaggatgccgatgtaggtggggtagcagcgccccccgcggatgctggcgcgcacgtagtcatacagctcgtgcgagggcgcgaggagccccgggcccaggttggtgcgactgggcttttcggcgcggtagacgatctggcgaaagatggcatgcgagttggaggagatggtgggcctttggaagatgttgaagtgggcgtgggggaggccgaccgagtcgcggatgaagtgggcgtaggagtcttgcagtttggcgacgagctcggcggtgacgaggacgtccagagcgcagtagtcgagggtctcctggatgatgtcatacttgagctggcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaggtgtccctgaccatgaccttgaggaactggtgcttgaaatcgatatcgtcgcagcccccctgctcccagagctggaagtccgtgcgcttcttgtaggcggggttgggcaaagcgaaagtaacatcgttgaaaaggatcttgcccgcgcggggcataaagttgcgagtgatgcggaaaggctggggcacctcggcccggttgttgatgacctgggcggcgagcacgatctcgtcgaaaccgttgatgttgtggcccacgatgtagagttccacgaatcgcgggcggcccttgacgtggggcagcttcttgagctcctcgtaggtgagctcgtcggggtcgctgagaccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcggtcccatttgagctggagggcgagatcgagggcgagctcgacgaggcggtcgtcccctgagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcgaccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcctcttcttcctcctccactaacatctcttctacttcctcctcaggcggtggtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctgccgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgttttgcaactttttttggaggccggaaatgaaactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagcaggcggcgcgagctgagcgaccaggagctgatgcacagcctgcagcgggccctgaccggggccgggaccgagggggagagctactttgacatgggcgcggacctgcactggcagcccagccgccgggccttggaggcggcaggcggtcccccctacatagaagaggtggacgatgaggtggacgaggagggcgagtacctggaagactgatggcgcgaccgtatttttgctagatgcaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacacgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtattcgcagcagcgagctgggcaggatcacgcgcccgcgcttgctgggcgaggaggagtacttgaatgactcgctgttgagacccgagcgggagaagaacttccccaataacgggatagagagcctggtggacaagatgagccgctggaagacgtatgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtaacccgttcgctcacctgcgcccccgcatcgggcgcatgatgtaagagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggtcagctgaaaaccatcatgcacaccaacatgcccaacgtgaacgagttcatgtacagcaacaagttcaaggcgcgggtgatggtctcccgcaagacccccaacggggtgacagtgacagatggtagtcaggatatcttggagtatgaatgggtggagtttgagctgcccgaaggcaacttctcggtgaccatgaccatcgacctgatgaacaacgccatcatcgacaattacttggcggtggggcggcagaacggggtcctggagagcgatatcggcgtgaagttcgacactaggaacttcaggctgggctgggaccccgtgaccgagctggtcatgcccggggtgtacaccaacgaggccttccaccccgatattgtcttgctgcccggctgcggggtggacttcaccgagagccgcctcagcaacctgctgggcattcgcaagaggcagcccttccaggagggcttccagatcatgtacgaggatctggaggggggcaacatccccgcgctcctggatgtcgacgcctatgagaaaagcaaggaggagagcgccgccgcggcgactgcagctgtagccaccgcctctaccgaggtcaggggcgataattttgccagccctgcagcagtggcagcggccgaggcggctgaaaccgaaagtaagatagtcattcagccggtggagaaggatagcaaggacaggagctacaacgtgctgccggacaagataaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccccgcggtggtgaaggaggaaagaaagccccgcaaaatcaagcgggtcaaaaaggacaaaaaggaagaagatgacgatctggtggagtttgtgcgcgagttcgccccccggcggcgcgtgcagtggcgcgggcggaaagtgcacccggtgctgagacccggcaccaccgtggtcttcacgcccggcgagcgctccggcagcgcttccaagcgctcctacgacgaggtgtacggggacgaggacatcctcgagcaggcggccgagcgcctgggcgagtttgcttacggcaagcgcagccgccccgccctgaaggaagaggcggtgtccatcccgctggaccacggcaaccccacgccgagcctcaagcccgtgaccctgcagcaggtgctgccgagcgcagcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagatccccacggagcccatggaaacgcagaccgagcccatgatcaagcccagcaccagcaccatggaggtgcagacggatccctggatgccatcggctcctagccgaagaccccggcgcaagtacggcgcggccagcctgctgatgcccaactacgcgctgcatccttccatcatccccacgccgggctaccgcggcacgcgcttctaccgcggtcatacaaccagccgccgccgcaagaccaccacccgccgccgccgtcgccgcacagccgctgcatctacccctgccgccctggtgcggagagtgtaccgccgcggccgcgcgcctctgaccctaccgcgcgcgcgctaccacccgagcatcgccatttaaactttcgcctgctttgcagatggccctcacatgccgcctccgcgttcccattacgggctaccgaggaagaaaaccgcgccgtagaaggctggcggggaacgggatgcgtcgccaccaccatcggcggcggcgcgccatcagcaagcggttggggggaggcttcctgcccgcgctgatccccatcatcgccgcggcgatcggggcgatccccggcattgcttccgtggcggtgcaggcctctcagcgccactgagacacttggaaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgacgcggaggagacgctgctgacgcacacggacgagccgcccccgtacgaggaggcggtgaaactgggtctgcccaccacgcggcccatcgcgcccctggccaccggggtgctgaaacccgaaagtaataagcccgcgaccctggacttgcctcctcccgcttcccgcccctctacagtggctaagcccctgccgccggtggccgtggcccgcgcgcgacccgggggctccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgctgtccgccagaaggaggagtgaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagcctggctcccaagggagcgcccaattccagccagtgggagcaaaaaaaggcaggcaatggtgacactatggaaacacacacatttggtgtggccccaatgggcggtgagaatattacaatcgacggattacaaattggaactgacgctacagctgatcaggataaaccaatttatgctgacaaaacattccagcctgaacctcaagtaggagaagaaaattggcaagaaactgaaagcttttatggcggtagggctcttaaaaaagacacaagcatgaaaccttgctatggctcctatgctagacccaccaatgtaaagggaggtcaagctaaacttaaagttggagctgatggagttcctaccaaagaatttgacatagacctggctttctttgatactcccggtggcacagtgaatggacaagatgagtataaagcagacattgtcatgtataccgaaaacacgtatctggaaactccagacacgcatgtggtatacaaaccaggcaaggatgatgcaagttctgaaattaacctggttcagcagtccatgcccaatagacccaactatattgggttcagagacaactttattgggctcatgtattacaacagtactggcaatatgggggtgctggctggtcaggcctcacagctgaatgctgtggtcgacttgcaagacagaaacaccgagctgtcataccagctcttgcttgactctttgggtgacagaacccggtatttcagtatgtggaatcaggcggtggacagttatgatcctgatgtgcgcattattgaaaaccatggtgtggaagacgaacttcccaactattgcttccccctggatgggtctggcactaatgccgcttaccaaggtgtgaaagtaaaaaatggtaacgatggtgatgttgagagcgaatgggaaaatgatgatactgtcgcagctcgaaatcaattatgcaagggcaacatttttgccatggaaattaacctccaagccaacctgtggagaagtttcctctactcgaacgtggccctgtacctgcccgactcttacaagtacacgccagccaacatcaccctgcccaccaacaccaacacttatgattacatgaacgggagagtggtgcctccctcgctggtggacgcctacatcaacatcggggcgcgctggtcgctggaccccatggacaacgtcaatcccttcaaccaccaccgcaacgcgggcctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgcctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccgtacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaaattgctacttgcatgatggctgagcccacaggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgtagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaacaatcaagacatgtaaaccgtgtgtgtatgtttaaaatatcttttaataaacagcactttaatgttacacatgcatctgagatgattttattttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagctgcagggcgcccagcaggtcgggcgcggagatcttgaaatcgcagttgggacccgcgttctgcgcgcgagagttgcggtacacggggttgcagcactggaacaccatcagggccgggtgcttcacgctcgccagcaccgccgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcacagccggcatcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagtttgccctcggcctcggtgcacccgtgcagccacagcgcgcacccggtgcactcccagttcttgtgggcgatctgggaatgcgcgtgcacgaacccttgcaggaagcggcccatcatggtcgtcagggtcttgttgctagtgaaggtcaacgggatgccgcggtgctcctcgttgatgtacaggtggcagatgcggcggtacacctcgccctgctcgggcatcagttggaagttggctttcaggtcggtctccacgcggtagcggtccatcagcatagtcatgatttccatgcccttctcccaggccgagacgatgggcaggctcatagggttcttcaccatcatcttagcactagcagccgcggccagggggtcgctctcatccagggtctcaaagctccgcttgccgtccttctcggtgatccgcaccggggggtagctgaagcccacggccgccagctcctcctcggcctgtctttcgtcctcgctgtcctggctgacgtcctgcatgaccacatgcttggtcttgcggggtttcttcttgggcggcagtggcggcggagatgcttgtggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccggcgacgagaagcagcagcagcagaatgaaagcttaaccgccccgccgcccagccccgcctccgacgcagccgcggtcccagacatgcaagagatggaggaatccatcgagattgacctgggctatgtgacgcccgcggagcatgaggaggagctggcagtgcgctttcaatcgtcaagccaggaagataaagaacagccagagcaggaagcagagaacgagcagagtcaggctgggctcgagcatggcgactacctccacctgagcggggaggaggacgcgctcatcaagcatctggcccggcaggccaccatcgtcaaggacgcgctgctcgaccgcaccgaggtgcccctcagcgtggaggagctcagccgcgcctacgagctcaacctcttctcgccgcgcgtgccccccaagcgccagcccaacggcacctgcgagcccaacccccgcctcaacttctacccggtcttcgcggtgcccgaggccctggccacctaccacatctttttcaagaaccaaaagatccccgtctcctgccgcgccaaccgcacccgcgccgacgccctcttcaacctgggtcccggcgcccgcctacctgatatcgcctccttggaagaggttcccaagatcttcgagggtctgggcagcgacgagactcgggccgcgaacgctctgcaaggagaaggaggaggagagcatgagcaccacagcgccctggtcgagttggaaggcgacaacgcgcggctggcggtgctcaaacgcacggtcgagctgacccatttcgcctacccggctctgaacctgcccccgaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctacccctcaaagtttggaagagcggcgcaagctcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccgccgcgactacatccgcgactgcgtctacctctacctctgccacacctggcagacgggcatgggcgtgtggcagcagtgtctggaggagcagaacctgaaagagctctgcaagctcctgcaaaagaacctcaagggtctgtggaccgggttcgacgagcggaccaccgcctcggacctggccgacctcatcttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgagggcgagggagccaaggggggtctgaaactcaccccggggctgtggacctcggcctacttgcgcaagttcgtgcccgaggattaccatcccttcgagatcaggttctacgaggaccaatcccagccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcggccccacagtagatgggacgagaccgggcgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaacttcccccgcaacatcttgcattactaccgtcacctccacagcccctactacttccaagaagaggcagcagcagcagaaaaagaccagaaaaccagctagaaaatccacagcggcggcagcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccagcaccgccatgagcaaagagattcccacgccttacatgtggagctaccagccccagatgggcctggccgccggcgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggcggtcctgactttggagagttcgtcctcgcagccccgctcgggcggcatcggcactctccagttcgtggaggagttcactccctcggtctacttcaaccccttctccggctcccccggccactacccggacgagttcatcccgaactttgacgccatcagcgagtcggtggacggctacgattgattaattaatcaactaaccccttacccctttaccctccagtaaaaataaagattaaaaatgattgaattgatcaataaagaatcacttacttgaaatctgaaaccaggtctctgtccatgttttctgtcagcagcacttcactcccctcttcccaactctggtactgcaggccccggcgggctgcaaacttcctccacactctgaaggggatgtcaaattcctcctgtccctcaatcttcatttttatcttctatcagatgtccaaaaagcgcgcgcgggtggatgatggcttcgaccccgtgtacccctacgatgcagacaacgcaccgactgtgcccttcatcaaccctcccttcgtctcttcagatggattccaagaaaagcccctgggggtgttgtccctgcgactggccgaccccgtcaccaccaagaatggggctgtcaccctcaagctgggggagggggtggacctcgacgactcgggaaaactcatctccaaaaatgccaccaaggccactgcccctctcagtatttccaacggcaccatttcccttaacatggctgcccctttttacaacaacaatggaacgttaagtctcaatgtttctacaccattagcagtatttcccacttttaacactttaggtatcagtcttggaaacggtcttcaaacttctaataagttgctgactgtacagttaactcatcctcttacattcagctcaaatagcatcacagtaaaaacagacaaaggactctatattaattctagtggaaacagagggcttgaggctaacataagcctaaaaagaggactgatttttgatggtaatgctattgcaacataccttggaagtggtttagactatggatcctatgatagcgatgggaaaacaagacccatcatcaccaaaattggagcaggtttgaattttgatgctaataatgccatggctgtgaagctaggcacaggtttaagttttgactctgccggtgccttaacagctggaaacaaagaggatgacaagctaacactttggactacacctgacccaagccctaattgtcaattactttcagacagagatgccaaatttaccctatgtcttacaaaatgcggtagtcaaatactaggcactgttgcagtagctgctgttactgtaggttcagcactaaatccaattaatgacacagtaaaaagcgccatagtattccttagatttgactctgacggtgtgctcatgtcaaactcatcaatggtaggtgattactggaactttagggaaggacagaccacccaaagtgtggcctatacaaatgctgtgggattcatgcccaatctaggtgcatatcctaaaacccaaagcaaaacaccaaaaaatagtatagtaagtcaggtatatttaaatggagaaactactatgccaatgacactgacaataactttcaatggcactgatgaaaaagacacaacacctgtgagcacttactccatgacttttacatggcagtggactggagactataaggacaagaatattacctttgctaccaactcctttactttctcctacatggcccaagaataaaccctgcatgccaaccccattgttcccaccactatggaaaactctgaagcagaaaaaaataaagttcaagtgttttattgattcaacagttttctcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgwgcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatggtttaaacgcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaataattgagtctgacaaaaggacttgatgtg

SEQ ID NO: 3 - DNA, 2661 bp, synthetic construct
  source 1..2661: mol_type = other DNA
  misc_feature 1..2661: note = Vector
ttaattaaccatcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgcgatcgctagcgacatcgatcacaagtttgtacaaaaaagcaggctccaccatgggaacccgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtacc

SEQ ID NO: 4 - DNA, 37490 bp, synthetic construct
  misc_feature 1..37490: note = Vector
  source 1..37490: mol_type = other DNA
gcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatcccgtcgagatatctagacccagctttcttgtacaaagtggtgatcgattcgacagatcgcgatcgcaagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgccggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggcggacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgccagctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaacccatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgagtttaaactcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagtttcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgwgcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatggttaattaaggatccttctatagtgtcacctaaatgtcgacggccaggcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagacggaaaaacatatcagaacgttcagcagtttatcgatgaaggcaattatacatccggcgacaaccatacgctgagagaccctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcaaactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttgcgccaagcttcttaatgaacatcaaaggcaataaaacatccgttgtcaaaaacagcatcctggagcaaggacagctgacagtcaactaataacagcaaaaagaaaatgccgatacttcattggcattttcttttatttctcaacaagatggtgaattgactagtgggtagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattgatccggaacccttaatataacttcgtataatgtatgctatacgaagttattaggtccctcgactatagggtcaccgtcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcaactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacggcgcgccaaagcttgcatgcctgcagccgcgtaacctggcaaaatcggttacggttgagtaataaatggatgccctgcgtaagcggggcacatttcattacctctttctccgcacccgacatagataataacttcgtatagtatacattatacgaagttatctagtagac

SEQ ID NO: 5 - protein, 1595 aa, synthetic construct
MSLLWGGVTVLAAMLIAGQVASVVFLVKGGGPGGGMQLFHLCLIISCSCPTVQASKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQCGGGSGGGMPLSYQHFRKLLLLDDEAGPLEEELPRLADEGLNRRVAEDLNLGNLNVSIPWTHKVGNFTGLASSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSARSQSEGPIFSAWWLQFRNSKPASDYALTHIVNLLEDWGPATEHGEHNIRIPRTPARVTGGVFLVDKNPHNTTESRLVVDFSQFSRGSTHVSWPKFAVPNLQSLTNLLSSNLSWLSLDVSAAFYHIPLHPAAMPHLLVGSSGLPRYVARLSSTSRNINYQHGTMQDLHDSCSRNLYVSLLLLYKTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKIKQCFRKLPVNRPIDWKVCQRIVGLLGFAAPFTQCGYPALMPLYACIQSKQAFTFSPTYKAFLCKQYLNLYPVARQRSGLCQVFADATPTGWGLAIGHRAMRGTFVAPLPIHTAELLAACFARSRSGAKLIGTDNSVVLSRKYTSFPWLLGCAANWILRGTSFVYVPSALNPAADPSAGRLGLYRPLLHLPFRPTTGRTSLYAVSPSVPSHLPDRVHFASPLHVAWRPPKSPNSNNPDWDFNPNKDHWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPNGSKGKRKRRAPVKQTLNFDLLKLAGDVESNPGPMDAMKRGLCCVLLLCGAVFVSPSQEIHARFRRMGGWSSKPRQGMGTNLSVPNPLGFFPDHQLDPAFGANSNNPDWDFNPNKDHWPEANQVGLEGGSGGMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 6 - protein, 1246 aa, synthetic construct
MSLLWGGVTVLAAMLIAGQVASVVFLVSKSGPPSGKSNSNNPDWDFNPNKDHWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPNGSKSGSKMPLSYQHFRKLLLLDDEAGPLEEELPRLADEGLNRRVAEDLNLGNLNVSIPWTHKVGNFTGLASSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSARSQSEGPIFSAWWLQFRNSKPASDYALTHIVNLLEDWGPATEHGEHNIRIPRTPARVTGGVFLVDKNPHNTTESRLVVDFSQFSRGSTHVSWPKFAVPNLQSLTNLLSSNLSWLSLDVSAAFYHIPLHPAAMPHLLVGSSGLPRYVARLSSTSRNINYQHGTMQDLHDSCSRNLYVSLLLLYKTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKIKQCFRKLPVNRPIDWKVCQRIVGLLGFAAPFTQCGYPALMPLYACIQSKQAFTFSPTYKAFLCKQYLNLYPVARQRSGLCQVFADATPTGWGLAIGHRAMRGTFVAPLPIHTAELLAACFARSRSGAKLIGTDNSVVLSRKYTSFPWLLGCAANWILRGTSFVYVPSALNPAADPSAGRLGLYRPLLHLPFRPTTGRTSLYAVSPSVPSHLPDRVHFASPLHVAWRPPSKSPGSGPPMQLFHLCLIISCSCPTVQASKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 7 - protein, 325 aa, synthetic construct
MDAMKRGLCCVLLLCGAVFVSPSQEIHARFRRMGGWSSKPRQGMGTNLSVPNPLGFFPDHQLDPAFGANSNNPDWDFNPNKDHWPEANQVGASKGGKSGMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 8 - protein, 843 aa, synthetic construct
MPLSYQHFRKLLLLDDEAGPLEEELPRLADEGLNRRVAEDLNLGNLNVSIPWTHKVGNFTGLASSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSARSQSEGPIFSAWWLQFRNSKPASDYALTHIVNLLEDWGPATEHGEHNIRIPRTPARVTGGVFLVDKNPHNTTESRLVVDFSQFSRGSTHVSWPKFAVPNLQSLTNLLSSNLSWLSLDVSAAFYHIPLHPAAMPHLLVGSSGLPRYVARLSSTSRNINYQHGTMQDLHDSCSRNLYVSLLLLYKTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKIKQCFRKLPVNRPIDWKVCQRIVGLLGFAAPFTQCGYPALMPLYACIQSKQAFTFSPTYKAFLCKQYLNLYPVARQRSGLCQVFADATPTGWGLAIGHRAMRGTFVAPLPIHTAELLAACFARSRSGAKLIGTDNSVVLSRKYTSFPWLLGCAANWILRGTSFVYVPSALNPAADPSAGRLGLYRPLLHLPFRPTTGRTSLYAVSPSVPSHLPDRVHFASPLHVAWRPP

SEQ ID NO: 9 - DNA, 2529 bp, synthetic construct
  source 1..2529: mol_type = other DNA
atgcctctgagctaccagcacttcagaaaactgctgctgctggacgacgaggccggacctcttgaggaagaactgcctagactggccgacgagggcctgaatagaagagtggccgaggacctgaacctgggcaacctgaatgtgtctatcccctggacacacaaagtgggcaacttcacaggcctggccagctctaccgtgcctgtgttcaatcccgagtggcagacccctagcttccctcacatccatctgcaagaggacatcatcaaccggtgccagcagtacgtgggccctctgaccgtgaacgagaagcggagactgaagctgatcatgcccgccagattctaccccaacctgaccaagtacctgcctctggacaagggcatcaagccttactaccctgagcacgccgtgaaccactacttcaagacccggcactacctgcacaccctgtggaaggccggcatcctgtacaagagagagacaaccagaagcgccagcttctgcggcagcccttactcttgggagcaagagctgcagcacggcagactggtgttccagacctctacaagacacggcgacgagtctttctgcagccagagcagcgggatcctgtctagatctcctgtgggaccttgtgtgcggagccagctgaaacagagcagactgggactgcagcctcagcagggatctctggctagaggcaagtctggcagaagcggcagcatcagagccagagtgcaccctaccaccagacggtcctttggcgtggaacctagcggcagcggccacatcgataatagcgccagcagcaccagcagctgtctgcatcagtctgctgtgcgcaagaccgcctacagccacctgagcacaagcaagagacagagcagctctggccacgccgtcgagctgcacaatatccctccaagcagcgccagaagccagagcgagggccctattttttctgcctggtggctgcagttcagaaacagcaagcccgccagcgactacgccctgacacacatcgtgaacctgctggaagattggggccctgccacagaacacggcgagcacaacatcagaatccccaggactcccgccagagtgaccggcggagttttcctggtggacaagaaccctcacaacaccaccgagagcaggctggtggtggacttcagccagtttagcagaggcagcacccacgtgtcctggcctaaattcgccgtgcctaacctgcagagcctgaccaatctgctgagcagcaacctgagctggctgagcctggatgtgtccgccgccttctatcacatcccactgcatcctgccgctatgccccatctgcttgtgggaagttctggcctgcctagatatgtggcccggctgtcctctaccagccggaacatcaattaccagcacgggaccatgcaggacctgcacgatagctgctcccggaacctgtacgttagcctgctcctgctgtacaaaaccttcggccggaagctgcacctgtactctcaccctatcatcctgggcttcagaaagatccccatgggcgtgggactgagcccatttctgctggcccagtttaccagcgccatctgcagtgttgtgcggagagcctttcctcactgcctggccttcagctacatggacgatgtggtgctgggcgccaagtctgtgcagcacctggaatctctgtttacctccatcaccaactttctgctgtccctgggaatccatctgaaccccaacaagacaaagagatggggctacagcctgaacttcatgggctacgtgatcggcagctggggcacactgcctcaagagcacatcgtgctgaagatcaagcagtgctttagaaagctgcccgtgaacagacccatcgactggaaagtgtgccagcggattgtgggcctgctgggatttgccgctcctttcacacagtgcggctaccccgctctcatgcctctgtacgcctgcatccagtccaagcaggccttcacattcagccctacctacaaggccttcctgtgcaagcagtacctgaatctgtaccccgtggctagacagagaagcggcctgtgtcaggtgttcgccgatgccacacctacaggatggggactcgccattggacacagggccatgagaggaacctttgtggcccctctgcctattcacaccgccgaattgctggctgcctgtttcgccagatctagaagcggcgccaagctgatcggcaccgacaattcagtggtgctgagccggaagtacaccagctttccatggctgctgggctgtgccgccaactggattctgagaggcaccagcttcgtgtacgtgcccagcgctcttaaccctgccgctgatcctagtgctggacggctgggactctacagaccactgctgcatctgcccttcagacccaccacaggcagaaccagcctgtatgccgtgtctcctagcgtgccaagccatctgcctgatcgggtgcactttgcctctccactgcatgtggcttggcggcctcca

SEQ ID NO: 10 - DNA, 2529 bp, synthetic construct
  source 1..2529: mol_type = other DNA
atgcctctgtcttatcaacactttagaaaattgctgcttcttgatgatgaagctggacctcttgaagaagaattgcctagacttgctgatgaaggacttaatagaagagttgctgaagatcttaatctgggaaatcttaatgtttctattccttggacacacaaagttggaaatttcacaggacttgcatcttctacagtgcctgtttttaatcctgaatggcaaacaccttcttttccacatattcatctgcaagaggatatcatcaatagatgtcaacaatatgttggaccactgacagttaatgagaagagaaggcttaaacttattatgcctgctagattctatcctaatcttacaaagtatttgcctctggataagggaatcaaaccttattatcctgaacatgctgtgaatcactactttaaaacaagacattatctgcatacactgtggaaagctggtattctttacaaaagagaaacaacaagatctgcttcattttgtggatctccatattcttgggaacaagaacttcaacatggtagacttgtttttcaaacatctacaagacatggggatgaatcattttgttctcaaagttctggaattctttctagatctcctgttggaccttgtgttagatctcaacttaaacaatctagacttggacttcaacctcaacaaggatctcttgctagaggaaaaagtggaagatctggatctattagagctagagttcatcctacaactagaagatcttttggagttgaaccttctggatctggacatattgataattctgcctcttctacatcttcttgtctgcatcaatctgctgttagaaagacagcttattctcatttgtctacttctaagagacaatcatcttctggacatgctgttgaacttcataatattcctccaagtagtgctagaagtcaatctgaaggaccaatattttcagcttggtggcttcaattcagaaattctaaacctgcttctgattatgctctgacacatatagttaatttgcttgaagattggggacctgctacagaacatggcgaacacaatattagaatacctagaactcctgctagagttacaggcggagtctttttggttgataagaatcctcataataccacagaatcaagacttgttgttgatttttcacagttttctagaggatctacacatgtttcttggcctaaatttgctgttccaaatcttcaatctcttacaaatttgctttcatctaatctttcttggctgtctcttgatgtttctgctgccttttatcatattcctcttcatcctgctgcaatgcctcatttgcttgttggatcatctggacttccaagatatgttgctagacttagctctacatctagaaatatcaattatcagcatggaacaatgcaggatcttcacgattcttgtagtaggaatctgtatgtttctttgcttctgctgtataagacatttggaagaaaacttcatctgtattctcaccctattattctgggttttagaaagattcctatgggagttggactttctccttttttgcttgctcaattcacatctgctatttgttctgttgttagaagggcttttcctcattgtcttgcattttcttatatggatgatgttgttcttggagctaaatctgttcaacatcttgaaagtctgtttacctctattactaattttctgctttctctgggaattcatctgaatccaaacaaaacaaagagatggggatattctcttaatttcatgggatatgttattggatcttggggaacacttcctcaagaacatatcgttttgaaaatcaagcaatgtttcagaaaactgcctgtgaatagacctattgattggaaagtttgtcaaagaattgtgggacttcttggatttgctgctccttttacacaatgtggatatcctgctcttatgccactttatgcttgtattcaatctaaacaggcttttacattttctccaacatacaaagcttttctgtgtaaacagtatctgaatctttatcctgtggctagacaaagatctggtctttgtcaagtttttgctgatgctacaccaacaggatggggacttgctattggacatagagctatgagaggaacatttgttgctccattgcctattcatacagctgaattgcttgctgcttgttttgctagatctagaagcggagcaaaacttattggtacagataatagtgttgtcctgagtagaaagtacacatcttttccatggttgttgggatgtgctgctaattggattcttagaggaacttcttttgtttatgttccttctgctcttaatcctgcagctgatccatctgctggtagattgggactgtatagaccacttcttcatttgccttttagaccaacaactggaagaacatctctttatgctgtttctccttctgttccatctcatttgcctgatagagttcattttgcttctccacttcatgttgcttggaggccacca

SEQ ID NO: 11 - protein, 29 aa, synthetic construct
MQLFHLCLIISCSCPTVQASKLCLGWLWG

SEQ ID NO: 12 - protein, 183 aa, synthetic construct
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 13 - protein, 226 aa, synthetic construct
MENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 14 - protein, 83 aa, synthetic construct
NSNNPDWDFNPNKDHWPEANQVGAGAFGPGFTPPHGGLLGWSPQAQGILTTVPAAPPPASTNRQSGRQPTPISPPLRDSHPQA

SEQ ID NO: 15 - protein, 59 aa, synthetic construct
MGGWSSKPRQGMGTNLSVPNPLGFFPDHQLDPAFGANSNNPDWDFNPNKDHWPEANQVG

SEQ ID NO: 16 - protein, 55 aa, synthetic construct
MQWNSTTFHQALLDPRVRGLYFPAGGSSSGTVNPVPTTASPISSIFSRTGDPAPN

(19) United States
(12) Patent Application Publication
(10) Pub. No.: US 2023/0310591 A1
(43) Pub. Date: Oct. 5, 2023

(54) Vaccine Boost Methods and Compositions

(71) Applicant: Vaccitech (UK) Limited, Oxford (GB)

(72) Inventors: Thomas Evans, Lexington, MA (US); Sarah Sebastian, Oxford (GB)

(73) Assignee: Vaccitech (UK) Limited, Oxford (GB)

(21) Appl. No.: 18/062,817

(22) Filed: Dec. 7, 2022

Related U.S. Application Data
(60) Provisional application No. 63/422,807, filed on Nov. 4, 2022.

(30) Foreign Application Priority Data
Dec. 7, 2021 (GB) GB 2117680.5
Jun. 22, 2022 (GB) GB 2209167.2

(51) Int. Cl. A61K 39/29; C12N 15/86

(52) U.S. Cl. CPC A61K 39/292; C12N 15/86; A61K 2039/545

(57) ABSTRACT

The invention relates to combinations, compositions, methods and dosage regimes for use in medicine, optionally wherein the use may be the treatment of chronic hepatitis B virus (HBV) infection or cancer, including inducing an improved immune response and improvement in the performance of therapeutic vaccines.

### CROSS-REFERENCE TO RELATED APPLICATIONS

This application claims priority to U.S. Provisional Application No. 63/422,807, filed Nov. 4, 2022, and to GB Application No. 2209167.2, filed Jun. 22, 2022, and to GB Application No. 2117680.5, filed Dec. 7, 2021, the contents of each of which are incorporated herein by reference in their entireties.

### SEQUENCE LISTING

This application contains a Sequence Listing which has been submitted electronically in XML format and is hereby incorporated by reference in its entirety. Said XML copy, created on Dec. 7, 2021, is named “2021-12-07_01304-0023-00US_SeqListing_ST26.xml” and is 106,769 bytes in size.

The present invention relates to combinations, compositions, methods and dosage regimes for use in medicine, optionally wherein the use may be the treatment of chronic hepatitis B virus (HBV) infection or cancer, including inducing an improved immune response and improvement in the performance of therapeutic vaccines.

Traditionally, vaccines have been based on whole inactivated or attenuated pathogens. However for many infectious diseases this approach is impractical. ‘Subunit vaccines’ have been developed which present an antigen to the immune system without introducing the whole infectious organism. However, this technique often induces only a weak immune response.

An alternative method has been developed which utilizes viral vectors for the delivery of antigens. Viruses replicate by transfecting their DNA into a host cell and inducing the transfected host cell to express viral genes and replicate the viral genome. This reproductive strategy has been harnessed to create vectored vaccines by creating recombinant, non-replicating viral vectors which carry one or more heterologous transgenes. Transfection or transduction of the recombinant viral genome into the host cell results in the expression of the heterologous transgene in the host cell. When the heterologous transgene encodes an antigen, for example, expression of the antigen within the host cell can result in its presentation to the host immune system and elicit a protective or therapeutic immune response by the host immune system. WO2012/172277 describes an adenovirus vector comprising a capsid derived from chimpanzee adenovirus AdY25, where the capsid encapsulates a nucleic acid molecule comprising an exogenous nucleotide sequence of interest.

For some infectious diseases such as HIV, malaria and tuberculosis, vaccine efforts have shifted to the stimulation of T-cell responses that have shown protection per se in both human and mouse models (Reyes-Sandoval et al, Eur J Immunol (2008) 38:732-741; Webster et al, Proc Natl Acad Sci USA (2005) 102:4836-4841).

More recently there has been increased interest in the development of additional viral vectors that could induce more potent T-cell responses. Reyes-Sandoval et al. (2010) Infection and Immunity p145-153 describes use of an adenoviral vector coding for a liver-stage antigen of the malaria parasite followed by modified vaccinia virus Ankara (MVA) to enhance protection against malaria, a so-called ‘prime-boost’ immunization strategy which increased the persistence of protection from malaria. T-cell inducing vaccines can also be used for therapeutic vaccination against cancer, tumours and chronic infectious diseases. WO2018/189522 describes a multi-HBV immunogen viral vector vaccine for therapeutic vaccination of chronic hepatitis B (CHB).

An aim of the present invention is to provide improved combinations, compositions, methods and dosage regimes for use in medicine, e.g. for use in the treatment of chronic HBV infection or cancer, including inducing an improved immune response and improvement in the performance of therapeutic vaccines.

### SUMMARY OF INVENTION

The inventors have found that administering a checkpoint inhibitor and a vaccine boost composition after administering a vaccine prime composition provides more effective treatment compared to administering a vaccine prime composition followed by a vaccine boost composition. The invention also provides a method of boosting an immune response in a subject in need thereof, the method comprising administering a vaccine boost composition and a checkpoint inhibitor, wherein the vaccine boost composition and the checkpoint inhibitor are administered at least 7 days after administration of a vaccine prime composition.

The invention provides a combination of compositions for use in a method of treatment. The combination comprises a vaccine prime composition, a vaccine boost composition and a checkpoint inhibitor. The method comprises administering the vaccine boost composition and the checkpoint inhibitor at least 7 days after administration of the vaccine prime composition. Kits are provided comprising a vaccine prime composition, a vaccine boost composition and a checkpoint inhibitor as a combined preparation for separate, simultaneous or sequential use in a method of treatment of a viral infection or cancer.

### BRIEF DESCRIPTION OF THE DRAWINGS

FIGS. 1A-1C Schemes showing the immunisation regimens of vaccine prime composition (prime) followed by checkpoint inhibitor and vaccine boost composition (boost), e.g, FIG. 1A, where the boost and checkpoint inhibitor are administered 7-56 days after the prime; FIG. 1B, where the checkpoint inhibitor may be administered prior to the boost, and FIG. 1C, where the checkpoint inhibitor and boost are given 28 days after the prime. The horizontal dotted lines indicate the timespan across which the vaccine boost composition (boost) or checkpoint inhibitor respectively may be given.

FIGS. 2A-2D ChAdOx1-HBV elicits an immune response. Total T cell responses to the HBV immunogen (Y-axis, IFNγ SFU/10⁶ peripheral blood mononuclear cells (PBMCs)) is plotted against time in days after vaccination (X-axis) for a healthy cohort (HC) (FIG. 2A) and patients with chronic HBV (CHB) with supressed HBV DNA on nucleos(t)ide therapy (FIG. 2B). FIG. 2C shows the response to HBV (Y-axis, IFNγ SFU/10⁶ PBMCs) at 28 days broken down by region (epitope) on the A axis, from the left: HBV core; HBV polymerase (pol); HBV surface pre-protein (Pre-S1); HBV surface. FIG. 2D shows cross reactivity of T-cell responses to both genotype C and genotype D peptides for cohorts HB and CHB.

FIGS. 3A-3E show plots of the average SFU/10⁶ PBMC against time (X-axis) (FIG. 3 A) and plots for individual patients. FIG. 3B shows core peptide pool response for Group 2, ChAdOx1-HBV (2.5×10¹⁰ viral particles) followed at d28 by MVA-HBV. FIG. 3C shows peptide response for Group 1 MVA-HBV (1×10⁸ plaque forming units (pfu)) followed at d28 by homologous MVA-HBV. FIG. 3D shows C peptide response for Group 2, ChAdOx1-HBV (2.5×10¹⁰ viral particles) followed at d28 by MVA-HBV. FIG. 3E shows D peptide response for Group 2, ChAdOx1-HBV (2.5×10¹⁰ viral particles) followed at d28 by MVA-HBV.

FIGS. 4A-4D show the changes in hepatitis B virus surface antigen measurement in subjects suffering chronic HBV infection at various time points before and following administration of a prime-boost vaccination regimen, where Group 1 received MVA-HBV alone, Group 2 received ChAdOx1-HBV followed by MVA HBV, Group 3 received ChAdOx1-HBV followed by MVA HBV with nivolumab at day 28 and Group 4 received ChAdOx1-HBV followed by MVA HBV with nivolumab at day 0 and day 28. FIG. 4 shows the log(10) response for each patient in each group (FIG. 4A=group 1; FIG. 4B=group 2, FIG. 4C=group 3, FIG. 4D=group 4) as a function of time. Samples were taken at day 0, 7, day 28, Day 35, Day 3 m (3 months).

FIG. 5. FIG. 5 shows the average group response to a prime-boost vaccination regimen where the response is measured by following the reduction in hepatitis B virus surface antigen measurement in subjects suffering chronic HBV infection, where Group 1 received MVA-HBV alone, Group 2 received ChAdOx1-HBV followed by MVA HBV, Group 3 received ChAdOx1-HBV followed by MVA HBV with nivolumab at day 28 and Group 4 received ChAdOx1-HBV followed by MVA HBV with nivolumab at day 0 and day 28. The least squared mean for each group (Y-axis) is plotted against time from vaccine prime (X-axis) determined using a model with timepoint as a discrete variable, with the baseline covariate, and using differences from the baseline.

FIGS. 6A-6B show the HBV surface antigen response by group. FIG. 6A shows the baseline HBsAg value of each subject on the x-axis plotted against the maximum drop in HBsAg recorded for that subject through month 9 (day 270). FIG. 6B shows the mean HBV surface antigen measurements for each group over time through month 9 (day 270). HBsAg is plotted on the Y-axis with units IU/mL.

FIGS. 7A-7D show the surface antigen responses by individual. Individual plots show results for those individuals from each group through months 3, 6 and 9 (FIG. 7A=group 1; FIG. 7B=group 2, FIG. 7C=group 3, FIG. 7D=group 4). HBsAg is plotted on the Y-axis with units IU/mL.

FIGS. 8A-8B show the CD8+ T cell interferon gamma (IFNγ) response (single cytokine response) vs. maximum drop in HBsAg for Core+Pol combined peptide pools and all HBV antigen combined peptide pools respectively measured using the ELISpot assay. FIGS. 8C-8D show the CD8+ T cell IFNγ and TNFa response (dual cytokine response) vs. maximum drop in HBsAg for Core+Pol combined peptide pools and all HBV antigen combined peptide pools respectively measured using the ELISpot assay. The maximum drop in HBsAg is plotted on the Y-axis with units log₁₀ IU/mL in all panels.

FIGS. 9A-9B show the CD4+ T-cell interferon gamma (IFNγ) response (single cytokine response) vs. maximum drop in HBsAg for Core+Pol combined peptide pools (FIG. 9A) and all HBV combined peptide pools (FIG. 9B) measured using the ELISpot assay. The Y-axis is on the same scale as FIGS. 8A-8D for ease of comparison.

FIGS. 10A-10B show the Day 35 total ELISPOT response measured in peripheral blood mononuclear cells (PBMCs) for Total HBV (all HBV combined peptide pools) vs. maximum drop in HBsAg (FIG. 10A), and the change in total ELISpot response measured in PBMCs between DO to Day 35 vs. maximum drop in HBsAg (FIG. 10B).

FIGS. 11A-11B show the Day 35 total ELISPOT response measured in PBMCs for Core+Pol combined peptide pools vs. maximum drop in HBsAg (FIG. 11A), and the change in total ELISpot response measured in PBMCs between DO to Day 35 vs. maximum drop in HBsAg (FIG. 11B).

FIG. 12 FIG. 12 provides data for the sum of IFNγ responses to peptide pools derived from all HBV antigens (Core+Pol+Pre-S+S) measured in PBMCs from each of groups 1-4 by ELISpot (Y axis: SFU/10⁶ PBMC), with data included for all patients with data through to at least the end of month 3. The prime and boost immunisation points are shown with black arrows.

FIG. 13. FIG. 13 provides data for the sum of IFNγ responses to peptide pools derived from all HBV antigens (Core+Pol+Pre-S+S) measured in PBMCs by ELISpot and plotted as fold change from baseline in each of groups 1-4 (Y axis: SFU/10⁶ PBMC fold change from baseline), with data included for all patients with data through to at least the end of month 3. The prime and boost immunisation points are shown with black arrows.

FIG. 14. FIG. 14 provides data as stacked bars representing the IFNγ responses to peptides derived from each of the HBV antigens (Core+Pol+Pre-S+S) as measured by ELISpot in each of groups 1-4 (Y axis: SFU/10⁶ PBMC fold). From the bottom, response to Core is shown in black ( [symbol] ), to Pol in dark grey ( [symbol] ) and to Pre-S+S in light grey ( [symbol] ).

FIG. 15 FIG. 15 provides data for the sum of IFNγ responses to peptides derived from each of the HBV antigens (Core+Pol+Pre-S+S) as measured by ELISpot and plotted as fold change from baseline in each of groups 1-4 (Y axis: SFU/10⁶ PBMC). From the bottom, response to Core is shown in black ( [symbol] ), to Pol in dark grey ( [symbol] ) and to Pre-S+S in light grey ( [symbol] ).

FIG. 16 FIG. 16 shows the ICS data for the sum of CD8+ T-cell IFNγ responses to HBV antigens (Core/Pol1/Pol2, Pol3/Pol4, Pre-S1-2+S) in each of groups 1-4 (Y axis: % CD8 IFNγ+), with data included for all patients with data through to at least the end of month 3. The prime and boost immunisation points are shown with black arrows.

FIG. 17 FIG. 17 provides the ICS data for the sum of CD4+ T-cell IFNγ responses to HBV antigens (Core/Pol1/Pol2, Pol3/Pol4, Pre-S1-2+S) in each of groups 1-4 (Y axis: % CD4 IFNγ+), with data included for all patients with data through to at least the end of month 3. The inoculation prime and boost immunisation points are shown with black arrows.

FIG. 18 FIG. 18 provides the same data as FIG. 17 but with the Y axis on the same scale as FIG. 16 for ease of comparison to the CD8+ T-cell IFNγ response. ICS data for the sum of CD4+ T-cell IFNγ responses to HBV antigens (Core/Pol1/Pol2, Pol3/Pol4, Pre-S1-2+S) is presented in each of groups 1-4 (Y axis: % CD4 IFNγ+), with data included for all patients with data through to at least the end of month 3. The prime and boost immunisation points are shown with black arrows.

FIG. 19 FIG. 19 provides the CD8 IFNγ ICS data as stacked bars representing the mean response to HBV antigens (Core+Pol1/2, Pol3/4, Pre-S1-2+S) in each of groups 1-4 (Y axis: % CD8 IFNγ+). From the bottom, response to core is shown in dark grey ( [symbol] ), to Pol1/2 in light grey ( [symbol] ), to Pol3/4 in white and to Pre-S1-2+S in black.

FIG. 20 FIG. 20 provides the CD4 IFNγ ICS data as stacked bars representing the mean response to HBV antigens (Core+Pol1/2, Pol3/4, Pre-S1-2+S) in each of groups 1-4 (Y axis: % CD4 IFNγ+). From the bottom, response to core is shown in dark grey ( [symbol] ), to Pol1/2 in light grey ( [symbol] ), to Pol3/4 in white and to Pre-S1-2+S in black.

FIG. 21 FIG. 21 provides the same CD4+ICS data as provided in FIG. 20, plotted on the same scale as the ICS date in FIG. 19 for ease of comparison to the CD8+ IFNγ response.

FIG. 22 SEQ ID NO: 5 is an antigen sequence encoded by ChAdOx1-HBV. SEQ ID NO: 6 and SEQ ID NO: 7 are antigen sequences encoded by MVA-HBV.

FIG. 23 SEQ ID NO: 8 is the HBV Pol protein sequence encoded by ChAdOx1-HBV and MVA-HBV. SEQ ID NO: 9 and SEQ ID NO: 10 are the nucleic acid sequences which encode the HBV Pol protein sequence in ChAdOx1-HBV and MVA-HBV respectively.

FIG. 24 SEQ ID NO: 11 (HBV Pre-Core), SEQ ID NO:12 (HBV-Core), SEQ ID NO: 13 (HBV-S), SEQ ID NO: 14 (HBV-NAPreS1), SEQ ID NO: 15 (HBV-CAPreS1) and SEQ ID NO: 16 (HBV-PreS2) are protein sequences encoded by the viral vectors ChAdOx1-HBV and MVA-HBV.

FIG. 25 FIG. 25 shows the HBV surface antigen response by group. The first graph shows the baseline HBsAg value of each subject on the x-axis plotted against the maximum drop in HBsAg recorded for that subject through month 9 (day 270) for group 2 and group 3. The second graph shows the mean HBV surface antigen measurements for each group over time through month 9 (day 270). HBsAg is plotted on the Y-axis with units IU/mL.

FIGS. 26A-26D show the surface antigen responses by individual. Individual plots show results for those individuals from each group through months 3, 6 and 9 (FIG. 26A=group 1; FIG. 26B=group 2, FIG. 26C=group 3, FIG. 26D=group 4). HBsAg is plotted on the Y-axis with units IU/mL.

FIG. 27A-27C present results of a mouse study investigating the administration of a checkpoint inhibitor (α-PD-1), when co-administered with the prime or with the boost immunization, or as a stand alone agent between prime and boost immunisations, in a heterologous prime-boost regimen. FIG. 27A shows average SFC/10⁶ splenocytes generated (Y-axis) in response to Pol 2 by group. FIG. 27B shows average SFC/10⁶ splenocytes generated (Y-axis) in response to Pre-S1/S2 by group. In FIGS. 27A and 27B, each dot represents an individual mouse response. In FIG. 27C, stacked bars representing average SFC/10⁶ splenocytes generated (Y-axis) to both Pol 2 (bottom) and Pre-S1/S2 (top) peptide pools, by groups.

### DETAILED DESCRIPTION

The invention is described in the claims. The invention provides a method for treating a subject in need thereof, wherein the method comprises administering a vaccine boost composition and a checkpoint inhibitor, wherein the vaccine boost composition and the checkpoint inhibitor are administered at least 7 days after administration of a vaccine prime composition. The method of treating can include boosting the immune response of a subject. Therefore the invention provides a method of boosting an immune response in a subject in need thereof.

The invention provides compositions for use in the methods of treatment of the invention, the methods comprising administering a vaccine boost composition and a checkpoint inhibitor at least 7 days after administration of a vaccine prime composition. The invention also provides use of a vaccine prime composition, a vaccine boost composition and a checkpoint inhibitor, optionally a PD-1 inhibitor, in the manufacture of a vaccine kit in a method of treatment, wherein the vaccine prime composition is administered at least 7 days before the vaccine boost composition and the checkpoint inhibitor, and optionally where the heterologous vaccine boost composition and the checkpoint inhibitor are administered on the same day.

The invention also provides a combination of compositions for use in a method of treatment, wherein the combination comprises a vaccine boost composition and a checkpoint inhibitor, the method comprising administering the vaccine boost composition and the checkpoint inhibitor at least 7 days after administration of the vaccine prime composition.

The invention also provides a composition for use in a method of treatment, wherein the composition comprises a vaccine prime composition, the method comprising administering the vaccine boost composition and the checkpoint inhibitor at least 7 days after administration of the vaccine prime composition.

The invention also provides a combination of compositions for use in a method of treatment, wherein the combination comprises a vaccine prime composition and a vaccine boost composition, the method comprising administering the vaccine boost composition and a checkpoint inhibitor at least 7 days after administration of the vaccine prime composition.

The invention relates to treatment methods and a method of inducing an immune response in an organism, such as a mammal, comprising the steps of exposing the organism to a priming composition, optionally comprising an adenoviral vector encoding one or more target antigens, and then boosting the immune response by administering a boosting composition, optionally comprising a pox viral vector encoding one or more target antigens, 7 days or more after the priming composition, and further boosting the immune response by administering a checkpoint inhibitor, such as a PD-1 inhibitor, 7 days or more after the priming composition. The method is preferably a heterologous prime-boost method.

Prime-Boost Regimen

A prime-boost regimen is method of vaccination involving the sequential administration of two vaccines, e.g. viral vectored vaccines, spaced by an interval of days or weeks.

Vaccine Prime Composition

A vaccine prime composition is the first administered vaccine composition, e.g. the first vaccine composition administered in a prime-boost regimen. The vaccine prime composition is preferably a viral vectored vaccine encoding one or more target antigens. The viral vector may be a non-replicating adenovirus. The non-replicating adenovirus may be of simian origin, such as chimpanzee adenovirus. The adenovirus may be the ChAdOx1 vector described in WO2012/172277, which is incorporated herein by reference. The viral vector may be a multi-HBV immunogen viral vector as described in WO2018/189522, which is incorporated herein by reference. Chimpanzee adenoviral vector ChAdOx1-HBV is a genetically modified (GM) non-replicating chimpanzee adenovirus vector encoding HBV polymerase, core, pre-S1 and pre-S2 polypeptide antigen consensus sequences from a group C genotype HBV. Chimpanzee adenoviral vector ChAdOx1-HBV also encodes pre-core and S antigen consensus sequences from a group C genotype HBV. The ChAdOx1 virus has been engineered to be replication deficient.

Vaccine Boost Composition

A vaccine boost composition is a vaccine composition that is administered after a vaccine prime composition, e.g. in a prime-boost regimen. The vaccine boost composition is administered at least 7 days after the vaccine prime composition. The vaccine boost composition preferably comprises a viral vectored vaccine encoding one or more target antigens. Preferably the one or more target antigens comprise the one or more target antigens encoded by the vaccine prime composition. In some embodiments the viral vectored vaccine does not replicate in the subject. The viral vector may be a non-replicating pox virus, such as Modified Vaccinia virus Ankara (MVA) as described in WOO1/21201, which is incorporated herein by reference. The viral vectored vaccine may be an RNA vectored vaccine. The RNA vectored vaccine may be a self-amplifying RNA. The vaccine boost composition may be the same as the vaccine prime composition (a homologous prime-boost method). The vaccine boost composition may be different from the vaccine prime composition (a heterologous prime-boost method). The vaccine boost composition may comprise the viral vector MVA-HBV. MVA-HBV is a GM poxvirus that is non-replicating in mammalian cells encoding the same polypeptide antigen consensus sequences as ChAdOx1-HBV.

Viral Vector

A viral vector may comprise a virus. The viral vector may be an attenuated viral vector. The viral vector may comprise an adenovirus, such as a human or simian adenovirus. In one embodiment, the viral vector comprises an adenovirus, such as a group E simian adenovirus, when used in a prime vaccine of a prime boost regime. The viral vector may comprise a group E simian adenovirus. The viral vector may comprise ChAdOx1 (a group E simian adenovirus, like the AdCh63 vector used safely in malaria trials) or ChAdOx2. The skilled person will be familiar with ChAdOx1 based viral vectors, for example from patent publication WO2012172277, which is herein incorporated by reference. The viral vector may comprise AdCh63. The viral vector may comprise AdC3 or AdH6. In one embodiment, the viral vector is a human serotype. In another embodiment, the viral vector comprises Modified Vaccinia Ankara (MVA).

In an alternative embodiment, the viral vector may comprise Adeno-associated virus (AAV) or Lentivirus. In another embodiment, the viral vector may comprise any of Vaccinia virus, fowlpox virus or canarypox virus (e.g. members of Poxviridae and the genus Avipoxvirus), or New York attenuated vaccinia virus (Tartaglia et al. Virology. 30 1992 May; 188(1):217-32, which is herein incorporated by reference). In another embodiment, the viral vector may comprise any of Herpes simplex virus, Cytomegalovirus (e.g. human cytomegalovirus), Measles virus (MeV), Sendai virus (SeV), Flavivirus (e.g. Yellow Fever Virus—17D), or alphavirus vectors, such as Sindibis virus (SINV), Venezuelan equine encephalitis virus, or Semliki forest virus.

Checkpoint Inhibitor

The checkpoint inhibitor may be a PD-1 inhibitor. The checkpoint inhibitor may be a PD-L1 inhibitor. Suitable PD-1 or PD-L1 inhibitors include small molecule inhibitors and anti-PD-1 or anti PD-L1 antibodies. Preferably the PD-1 inhibitor is an anti-PD-1 antibody, such Keytruda (pembrolizumab), Opdivo (nivolurab), Liblayo (cemiplimab), Tecentriq (atezolizumab), Bavencio (avelumab), and Imfinzi (durvalumab), most preferably nivolumab. The checkpoint inhibitor may be administered at a low dose, preferably at a dose below the dose approved for use, optionally where the dose is at least 1/10 of the dose approved for use, e.g. for treatment of cancer. The dose may be around 0.3 mg/kg. The check point inhibitor, e.g. an anti-PD-1 antibody such as nivolumab, may be administered by intravenous infusion. A pharmaceutical composition is provided comprising low dose of anti-PD-1 antibody for use in the methods of the invention. A low dose may be less than 50%, e.g. 10% to 50% of the dose recommended for treatment of cancer. A low dose may be from 0.1 mg/kg to 1.5 mg/kg. A low dose may be from 0.2 mg/kg to 1.0 mg/kg. Preferably the checkpoint inhibitor is not administered prior to the administration of the vaccine prime composition. Preferably the method comprises not administering the checkpoint inhibitor until at least 7 days, or more preferably 28 days, after administration of the vaccine prime composition. Preferably the first dose of the checkpoint inhibitor is administered at least 7 days, or more preferably 28 days, after administration of the vaccine prime composition. Preferably the checkpoint inhibitor is administered as a single dose. Preferably the checkpoint inhibitor is administered on the same day as the vaccine boost composition. Preferably the checkpoint inhibitor is administered at the same time as the vaccine boost composition. Advantageously, administering at the same time includes administering during the same patient procedure. Providing the checkpoint inhibitor and the vaccine composition at the same time, so that only one visit is required, should improve patient compliance and yield better outcomes.

Dosage Regimen

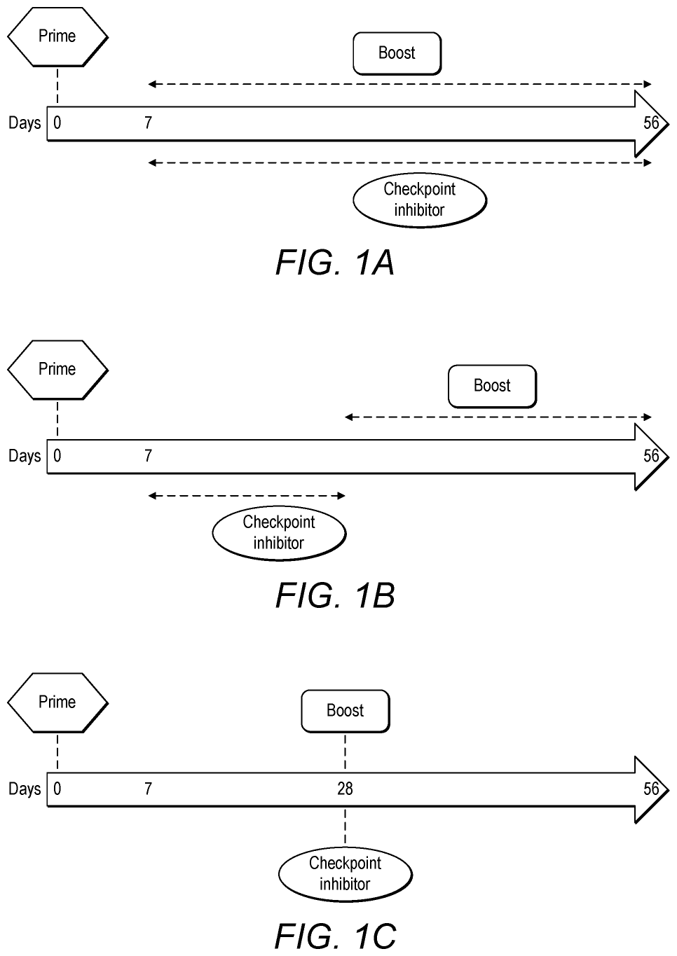

The vaccine prime composition and the vaccine boost composition are administered at least 7 days apart. The immune response induced is further boosted if a checkpoint inhibitor is also administered at least 7 days after the vaccine prime composition. The vaccine boost composition and the checkpoint inhibitor may be administered up to 56 days after the vaccine primer (FIG. 1A). The vaccine boost composition and the checkpoint inhibitor may be administered sequentially, with the checkpoint inhibitor being administered before the vaccine boost composition. The checkpoint inhibitor may be administered at least 7 days after the vaccine prime composition. The checkpoint inhibitor may be administered at about 7 days after the vaccine prime composition and the vaccine boost composition may be administered at least 1 day, or at least 7 days, or at least 14 days, or at least 21 days after the checkpoint inhibitor. The checkpoint inhibitor may be administered at 7-35 days, 7-28 days, 7-21 days, or 7-14 days after the vaccine prime composition. The checkpoint inhibitor may be administered at least 14 days after the vaccine prime composition. The vaccine boost composition may be administered at least 1 day, or at least 7 days, or at least 14 days, or at least 21 days after the checkpoint inhibitor. The vaccine boost composition may be administered about 28 days after the vaccine prime composition (FIG. 1B). Thus the checkpoint inhibitor may be administered at about 7 days after the vaccine prime composition and the vaccine boost composition may be administered at about 28 days after the vaccine prime composition. Alternatively the vaccine boost composition and the checkpoint inhibitor may be administered on the same day, such as at about 7 days, 14 days, 21 days or 28 days, preferably about 28 days after the vaccine prime composition (FIG. 1C). Administration at about 7 days after the vaccine prime composition can include administration at 7 days, 8 days, or 9 days after the vaccine prime composition. The vaccine boost composition and/or checkpoint inhibitor may be administered at least 84 days after the vaccine prime composition. The method and dosage regimes may include more than one administration of the vaccine boost composition, such as administration of a further (second) dose of a vaccine boost composition on up to day 84, preferably up to day 56, and optionally a further (third) dose of a vaccine boost composition on up to day 84. The method and dosage regimes may include more than one administration of the checkpoint inhibitor, such as administration of a further (second) dose of checkpoint inhibitor on up to day 84, preferably up to day 56, and optionally a further (third) dose of checkpoint inhibitor on up to day 84. The method and dosage regimes may administration of a dose or a further (second) dose of checkpoint inhibitor after administration of the vaccine boost composition, such as at about 7 days, 14 days, 21 days or 28 days, preferably about 28 days after the vaccine boost composition. Administration at about 7 days after the vaccine boost composition can include administration at 7 days, 8 days, or 9 days after the vaccine boost composition. A single dose of the checkpoint inhibitor may be administered. The checkpoint inhibitor and vaccine boost composition may be administered substantially at the same time. For example, they may be administered on the same day. Vaccine boost composition may be administered within an hour of administration of checkpoint inhibitor and vice versa. The method of treatment may consist of administering a vaccine boost composition and a checkpoint inhibitor at least 7 days after administration of a vaccine prime composition, preferably about 28 days after administration of a vaccine prime composition.

Method of Treatment

The invention provides methods of treatment, compositions for use in a method of treatment and dosage regimes. Treatment can mean a cure of the disease, e.g. HBV or cancer, an alleviation of symptoms or a reduction or slowing of severity in the disease or symptoms of the disease.

Composition

The pharmaceutical compositions of the invention which include a vaccine composition, such as a vaccine prime composition or a vaccine boost composition, and a checkpoint inhibitor composition, may comprise one or more additional active ingredients, an adjuvant, a pharmaceutically acceptable carrier, diluent and/or excipient.

Suitable carriers and/or diluents are well known in the art and include pharmaceutical grade starch, mannitol, lactose, magnesium stearate, sodium saccharin, talcum, cellulose, glucose, sucrose, saccharose (or other sugar), magnesium carbonate, gelatin, oil, alcohol, detergents, emulsifiers or water (preferably sterile). The composition may be a mixed preparation of a composition or may be a combined preparation for simultaneous, separate or sequential use (including administration).

Suitable adjuvants are well known in the art and include incomplete Freund's adjuvant, complete Freund's adjuvant, Freund's adjuvant with MDP (muramyldipeptide), alum (aluminium hydroxide), alum plus Bordatella pertussis and immune stimulatory complexes (ISCOMs, typically a matrix of Quil A containing viral proteins).

The composition according to the invention for use in the aforementioned indications may be administered by any convenient method, for example by oral (including by inhalation), parenteral (including by injection and by infusion), mucosal (e.g. buccal, sublingual, nasal), rectal or transdermal administration and the compositions adapted accordingly.

A liquid formulation will generally consist of a suspension or solution of the compound or physiologically acceptable salt in a suitable aqueous or non-aqueous liquid carrier(s) for example water, ethanol, glycerine, polyethylene glycol or oil. The formulation may also contain a suspending agent, preservative, flavouring or colouring agent.

Typical parenteral compositions consist of a solution or suspension of the compound or physiologically acceptable salt in a sterile aqueous or non-aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

The pharmaceutical composition is preferably sterile. It is preferably pyrogen-free. It is preferably buffered e.g. at between pH 6 and pH 8, generally around pH 7. Preferably, the composition is substantially isotonic with humans. Preferably, the pharmaceutical compositions of the present invention deliver an immunogenically or pharmaceutically effective amount of a viral vector or of a checkpoint inhibitor to a patient. In general, a pharmaceutically effective dose of a ChAdOx1-vectored vaccine composition comprises 1×10⁷ to 1×10¹² viral particles, preferably 2×10⁸ to 1×10¹¹ particles. More preferably, a pharmaceutically effective dose of a ChAdOx1-vectored vaccine composition comprises 2.5×10¹⁰ viral particles. In general, a pharmaceutically effective dose of an MVA-vectored vaccine composition comprises 1×10⁵ to 1×10¹¹ plaque forming units (pfu), preferably 1×10⁷ to 1×10¹ pfu. More preferably, a pharmaceutically effective dose of an MVA-vectored vaccine composition comprises 1×10⁸ pfu.

Conveniently the vaccine prime composition, vaccine boost composition or checkpoint inhibitor composition is in unit dose form such as a capsule or ampoule.

Conditions

Preferably, the compositions of the present invention, preferably when administered according to the method or dosage regime of the present invention, are capable of eliciting, inducing or boosting an antigen-specific immune response. Preferably, the immune response is a strong T cell immune response, for example a strong CD8+ T cell response and optionally a CD4+ T cell response. Preferably, the T cell immune response is a protective T cell immune response. Preferably, the T cell immune response is long lasting and persists for at least 1, 2, 5, 10, 15, 20, 25 or more years.

The compositions and dosage regimens of the invention may be used to treat conditions which require induction of a T-cell response to treat the condition, such as hepatitis B. The compositions and dosage regimens preferably may be used to treat conditions which require induction of a CD8+ T cell response. For example, induction of a CD8+ T cell response to HBV can be used to treat chronic hepatitis B (CHB). Preferably the T cell response, optionally the CD8+ T cell response, is induced by administering a vaccine prime composition, e.g. including a replication incompetent adenoviral vector, followed by administering a vaccine boost composition, e.g. including an attenuated poxvirus vector. More preferably administration of the vaccine prime composition is also followed by administering a check point inhibitor. Conditions which may be treated include cancer, conditions (E.g. including cancer) caused by viruses such as hepatitis B virus (HBV), herpes simplex virus, Epstein Barr virus, Shingles (varicella-zoster virus) and human papillomavirus, and conditions caused by bacteria such as tuberculosis and Chlamydia. The compositions, dosage regimes and methods can be used to treat chronic hepatitis B (CHB) (and infection with HBV). The compositions, dosage regimes and methods can be used to treat cancer, including prostate cancer, cancers which express melanoma antigen gene (MAGE), also known as MAGE+ cancers, and cancers which express New York Esophogeal Squamous Cell Carcimoma-1 (NY-ESO-1) which is also known as cancer-testis antigen 1B (CTAG1B).

Subject

The subject being treated using the method of treatment may be in need of an antigen-specific CD8+ T cell response, e.g. a patient suffering from a viral infection such as chronic HBV infection, herpes simplex virus (HSV), Epstein Barr virus (EBV), varicella-zoster virus (VZV), human papilloma virus (HPV), Middle East Respiratory Syndrome-related coronavirus (MERS-CoV), a bacterial infection such as Mycobacterium tuberculosis and Chlamydia trachomatis or cancer, such as prostate cancer, cancers which express melanoma antigen gene (MAGE), also known as MAGE+ cancers, and cancers which express New York Esophogeal Squamous Cell Carcimoma-1 (NY-ESO-1) which is also known as cancer-testis antigen 1B (CTAG1B). The subject being treated using the method of treatment may suffer from chronic infection such as a chronic HBV infection. The subject being treated using the method of treatment may have undergone therapy for the condition being treated, such as antiviral therapy, prior to administering the vaccine prime composition. The subject may have undergone therapy for at least a month, at least 3 months, at least 6 months, at least 9 months, or least 12 months prior to administering the vaccine prime composition, most preferably at least 12 months.

The subject is preferably virally suppressed. Virally suppressed includes subjects that have been routinely administered antiviral agents directed to the virus which is suppressed, and for whom the viral load is undetectable. The viral load can be measured by measuring the copies of viral DNA. The viral load can be considered undetectable when viral DNA <40 copies/mL. Subjects with chronic Hepatitis B may have undetectable viral load. Subjects with chronic hepatitis B may have Hepatitis B surface antigen (HBsAg) levels <4000 IU/mL. A subject's Hepatitis B surface antigen (HBsAg) levels may be reduced by treatment with agents that reduce such levels, for example siRNA agents. Subjects with chronic hepatitis B may have Hepatitis B surface antigen (HBsAg) levels <1000 IU/mL, <500 IU/mL, <400 IU/mL, <300 IU/mL, <200 IU/mL, <100 IU/mL, <50 IU/mL or <20 IU/mL. In one embodiment subjects with chronic hepatitis B have Hepatitis B surface antigen (HBsAg) levels <1000 IU/mL. In one embodiment subjects with chronic hepatitis B have Hepatitis B surface antigen (HBsAg) levels <100 IU/mL. In one embodiment subjects with chronic hepatitis B have Hepatitis B surface antigen (HBsAg) levels <50 IU mL.

The subject may have Hepatitis B virus genotype A, B, C, D or E, such as Hepatitis B virus genotype C. The subject may have Hepatitis B virus genotype A, B, C, D, E, F or G. The subject may have Hepatitis B virus genotype B, C, or D, such as Hepatitis B virus genotype B or C, or genotype C or D.

Target Antigens

An antigen is a protein or polypeptide of interest. As used herein the term antigen encompasses one or more epitopes from an antigen and includes the parent antigen, and fragments and variants thereof. The antigen may be a pathogen-derived antigen, such as an antigen selected from the group consisting of hepatitis B virus (HBV), herpes simplex virus (HSV), Epstein Barr virus (EBV), varicella-zoster virus (VZV), human papilloma virus (HPV), Mycobacterium tuberculosis and Chlamydia trachomatis. A suitable antigen for HBV may comprise the inactivated polymerase (Pol), core, and the S region, or fragments thereof, e.g. from genotype C HBV.

The antigen may be a self-antigen. The antigen may be a neoantigen. Suitable antigens include antigens expressed by tumour cells which allow the immune system to differentiate between tumour cells and other cell types. Suitable self-antigens include antigens that are either inappropriate for the cell type and/or its environment, or are only normally present during the organisms' development (e.g. foetal antigens). For example, GD2 is normally only expressed at a significant level on the outer surface membranes of neuronal cells, where its exposure to the immune system is limited by the blood-brain barrier. However, GD2 is expressed on the surfaces of a wide range of tumour cells including small-cell lung cancer, neuroblastoma, melanomas and osteosarcomas. Other suitable self-antigens include cell-surface receptors that are found on tumour cells but are rare or absent on the surface of healthy cells. Such receptors may be responsible for activating cellular signalling pathways that result in the unregulated growth and division of the tumour cell. For example, ErbB2 is produced at abnormally high levels on the surface of breast cancer tumour cells. Preferably, the self-antigen comprises a tumour-associated antigen (TAA).

Chimpanzee adenoviral vector ChAdOx1-HBV is a genetically modified (GM) non-replicating chimpanzee adenovirus vector encoding HBV consensus polymerase, core, pre-S1 and pre-S2 polypeptide antigen sequences from a group C genotype. ChAdOx1-HBV also encodes pre-core, and S polypeptide antigen sequences from a group C genotype. The ChAdOx1 virus has been engineered to be replication deficient.

The methods and compositions herein may be used to treat or to induce and/or boost an immune response against Hepatitis B virus, preferably chronic HBV (CHB).

The prime-boost regimen can produce a cross reactive response. A prime boost regimen comprising ChAdOx1-HBV and MVA-HBV, comprising polypeptide antigen sequences from a group C genotype HBV, can be used to treat or to induce and/or boost an immune response HBV group C and one or more further HBV genotypes, such as group B and group D.

Boosting an Immune Response

The term boost as used herein relates to increasing the immune response. The immune response can be measured for example by measuring levels of antigen-specific antibodies or T-cells in the blood of a subject using ELISA or ELISpot assays respectively. Alternatively, an immune response can be measured by measuring the levels of a target antigen in the blood of the subject following administration of the compositions or combinations of the invention according to the methods of the invention.

Kits

A kit is provided for use in inducing an immune response in an organism, comprising a vaccine prime composition and a vaccine boost composition which are administered separately. A kit may also comprise a checkpoint inhibitor, such as an anti-PD-1 antibody.

Kits are provided comprising two or more of the compositions described herein, such as a vaccine prime composition, a vaccine boost composition and a checkpoint inhibitor, as a combined preparation for separate, simultaneous or sequential use in a method of treatment of a viral infection or cancer. A kit may comprise a vaccine prime composition and a vaccine boost composition. The kits may be used with the methods of treatment described herein. Preferably the kits may be used in methods of treatment of chronic HBV infection described herein.

Preferably the kits are used in methods comprising administering the vaccine boost composition and the checkpoint inhibitor at least 7 days after administration of the vaccine prime composition.

Viral Vector

In one embodiment, the viral vector may comprise nucleic acid comprising the sequence of SEQ ID NO: 1 and 2 (ChAdOx1) or a variant thereof. A variant of SEQ ID NO: 1 and 2 may comprise a sequence having at least 70%, 75%, 80%, 85%, 90%, 95%, 98%, 99%, or 99.5% identity with SEQ ID NO: 1 and 2. The variant of SEQ ID NO: 1 and 2 may encode a viral vector that substantially retains the function of the viral vector of SEQ ID NO: 1 and 2 (ChAdOx1).

In one embodiment, the viral vector may comprise nucleic acid comprising the sequence of SEQ ID NO: 3 and 4 (ChAdOx2) or a variant thereof. A variant of SEQ ID NO: 3 and 4 may comprise a sequence having at least 70%, 75%, 80%, 85%, 90%, 95%, 98%, 99%, or 99.5% identity with SEQ ID NO: 3 and 4. The variant of SEQ ID NO: 3 and 4 may encode a viral vector that substantially retains the function of the viral vector of SEQ ID NO: 3 and 4 (ChAdOx2).

Antigens

HBV Antigen

The viral vector preferably encodes multiple HBV antigens, such as the Core, Polymerase and Surface. The antigens may be organised in an immunogen expression cassette. The viral vector may encode a protein sequence comprising SEQ ID NO: 5 (antigen sequence in ChAdOx1-HBV), SEQ ID NO: 6 (antigen sequence 1 in MVA-HBV) or SEQ ID NO: 7 (antigen sequence 2 in MVA-HBV) or a variant thereof. A variant of SEQ ID NO: 5, 6 or SEQ ID NO: 7 may comprise a sequence having at least 70%, 75%, 80%, 85%, 90%, 95%, 98%, 99%, or 99.5% identity with SEQ ID NO: 5, 6 or 7 respectively. The vaccine prime composition may comprise a viral vector (such as ChAdOx1) comprising SEQ ID NO: 5 or a variant thereof. The vaccine boost composition may comprise a viral vector (such as MVA) comprising SEQ ID NO: 6 and SEQ ID NO: 7.

HBV Polymerase

The HBV polymerase (Pol) is a modified (or mutated) HBV polymerase and may comprise or consist of a truncated HBV polymerase. In particular, the mutation to wild-type HBV polymerase to substantially remove polymerase function may comprise a sequence encoding a truncated HBV polymerase. Alternatively or additionally, the mutation comprises one or more point mutations to the encoded HBV polymerase sequence. The modification may comprise one or more amino acid substitutions, deletions or additions in the encoded HBV polymerase sequence. In one embodiment, the modified HBV polymerase (Pol) is not a truncated form of HBV polymerase (i.e. it is full length relative to wildtype HBV polymerase).

The modified HBV polymerase (Pol) may comprise or consist of the sequence of SEQ ID NO: 8 or a variant thereof. A variant of SEQ ID NO: 8 may comprise a sequence having at least 70%, 75%, 80%, 85%, 90%, 95%, 98%, 99%, or 99.5% identity with SEQ ID NO: 8. The variant of SEQ ID NO: 8 may substantially retain the immunogenicity of SEQ ID NO: 8. The variant of SEQ ID NO: 8 may substantially retain the tertiary structure of SEQ ID NO: 8.

The modified HBV polymerase (Pol) may comprise or consist of nucleic acid sequence of SEQ ID NO: 9 or SEQ ID NO: 10 or a variant of. A variant of SEQ ID NO: 9 and 10 may comprise a sequence having at least 70%, 75%, 80%, 85%, 90%, 95%, 98%, 99%, or 99.5% identity with SEQ ID NO: 9 and 10.

HBV Core

The HBV Core may comprise or consist of a full length wild-type HBV Core sequence, or a variant thereof. The HBV Core variant may comprise or consist of a truncated HBV Core sequence. The HBV Core may or may not comprise HBV Pre-Core (SEQ ID NO: 11: MQLFHLCLIISCSCPTVQASKLCLGWLWG) or a variant thereof. The HBV Core may comprise or consist of the sequence of SEQ ID NO: 12 or a variant thereof (SEQ ID 12: MDIDPYKEFGASVELLSFLPSDFFPSIRDLLDTASALYREALESPEHCSPHHTALRQAILCWGEL MNLATWVGSNLEDPASRELVVSYVNVNMGLKIRQLLWFHISCLTFGRETVLEYLVSFGVWIRTP PAYRPPNAPILSTLPETTVVRRRGRSPRRRTPSPRRRRSQSPRRRRSQSRESQC). A variant of SEQ ID NO: 11 or SEQ ID 12 may comprise a sequence having at least 70%, 75%, 80%, 85%, 90%, 95%, 98%, 99%, or 99.5% identity with SEQ ID NO: 11 or SEQ ID NO: 12 respectively.

HBV Surface Antigen (HbsAg)

The skilled person will understand that PreS1 and PreS2 are components of the Large (L) form of HBV surface protein (e.g. L form=PreS 1+PreS2+S). The medium (M) form of HBV surface protein has PreS2+S. The HbsAg may comprise or consist of a full length wild-type HbsAg sequence, or a variant thereof. The HbsAg variant may comprise or consist of a truncated HbsAg sequence. In another embodiment, the HbsAg may comprise the surface antigen (S) without PreS1 and/or PreS2. The HbsAg may comprise or consist of the sequence of SEQ ID NO: 13 or a variant thereof. (SEQ ID NO: 13: MENTTSGFLGPLLVLQAGFFLLTRILTIPQSLDSWWTSLNFLGGAPTCPGQNSQSPTSNHSPTS CPPICPGYRWMCLRRFIIFLFILLLCLIFLLVLLDYQGMLPVCPLLPGTSTTSTGPCKTCTIPAQGT SMFPSCCCTKPSDGNCTCIPIPSSWAFARFLWEWASVRFSWLSLLVPFVQWFVGLSPTVWLS VIWMMWYWGPSLYNILSPFLPLLPIFFCLWVYI)

A variant of SEQ ID NO: 13 may comprise a sequence having at least 70%, 75%, 80%, 85%, 90%, 95%, 98%, 99%, or 99.5% identity with SEQ ID NO: 13.

HBV PreS1

The HBV PreS1 may comprise or consist of a full length wild-type HBV PreS1 sequence, or a variant thereof. The HBV PreS1 variant may comprise or consist of a truncated HBV PreS1 sequence, for example CΔPreS1 (SEQ ID NO: 14: MGGWSSKPRQGMGTNLSVPNPLGFFPDHQLDPAFGANSNNPDWDFNPNK DHWPEANQVG) or NAPreS1 (SEQ ID NO: 15:NSNNPDWDFNPNKDHWPEANQVGAGAFGPGFTPPHGGLLGWSPQAQGIL TTVPAAPPPASTNRQSGRQPTPISPPLRDSHPQA) described herein (CΔPreS1 refers to C-terminal truncated PreS1 and NΔPreS1 refers to N-terminal truncated PreS1). In one embodiment, the viral vector may encode both NΔPreS1 and CΔPreS1.

HBV PreS2

The HBV PreS2 may comprise or consist of a full length wild-type HBV PreS2 sequence, or a variant thereof. The HBV PreS2 variant may comprise or consist of a truncated HBV PreS2 sequence. The HBV PreS2 may comprise or consist of the sequence of SEQ ID NO: 16 or a variant thereof. (SEQ ID 16: MQWNSTTFHQALLDPRVRGLYFPAGGSSSGTVNPVPTTASPISSI FSRTGDPAPN). A variant of SEQ ID NO: 16 may comprise a sequence having at least 70%, 75%, 80%, 85%, 90%, 95%, 98%, 99%, or 99.5% identity with SEQ ID NO: 16.

Preferably the viral vector encodes the HBV antigens pre-Core, Core, Pol, Pre-S1 (as NΔPreS1 and CΔPreS1), Pre-S2 and S.

Where the one or more antigens encoded by the one or more vectors in the composition are from a pathogen, the medicament may be intended/used to treat or to confer protection from the infection and/or disease caused by the pathogen from which the antigen of interest is derived. Alternatively, there the antigen is a cancer antigen or an antigen associated with a particular disease, the medicament may be intended/used to confer protection from and/or to treat the cancer of the particular disease from which the antigen is derived.

### Specific Embodiments of the Invention

A1. A combination of compositions for use in a method of treatment, wherein the combination comprises a vaccine prime composition, a vaccine boost composition and a checkpoint inhibitor, the method comprising administering the vaccine boost composition and the checkpoint inhibitor at least 7 days after administration of the vaccine prime composition.

A2. A combination of compositions for use in a method of treatment, wherein the combination comprises a vaccine boost composition and a checkpoint inhibitor, the method comprising administering the vaccine boost composition and the checkpoint inhibitor at least 7 days after administration of the vaccine prime composition.

A3. A composition for use in a method of treatment, wherein the composition comprises a vaccine prime composition, the method comprising administering the vaccine boost composition and the checkpoint inhibitor at least 7 days after administration of the vaccine prime composition.

A4. A combination of compositions for use in a method of treatment, wherein the combination comprises a vaccine prime composition, and a vaccine boost composition, the method comprising administering the vaccine boost composition and a checkpoint inhibitor at least 7 days after administration of the vaccine prime composition.

A5. The composition or combination of compositions for use as described in any one of embodiments A1 to A4, wherein the use in a method of treatment is the treatment of chronic hepatitis B virus (HBV) infection or cancer.

A6. The composition or combination of compositions for use as described in any one of embodiments A1 to A5, wherein the checkpoint inhibitor is a PD-1 inhibitor, preferably wherein the PD-1 inhibitor is an antibody, even more preferably wherein the antibody is nivolumab.

A7. The composition or combination of compositions for use as described in any one of embodiments A1 to A6, wherein the vaccine boost composition comprises a nucleic acid sequence encoding a target antigen against which an immune response is desired, preferably wherein the target antigen is derived from HBV.

A8. The composition or combination of compositions for use as described in any one of embodiments A1 to A7, wherein the vaccine boost composition comprises a viral vectored vaccine.

A9. The composition or combination of compositions for use as described in embodiment A8, wherein the viral vectored vaccine is a poxvirus, preferably wherein it is Modified Vaccinia Virus Ankara (MVA).

A10. The composition or combination of compositions for use as described in any one of embodiments A1 to A7, wherein the vaccine boost composition comprises an RNA vectored vaccine, optionally wherein the RNA vectored vaccine is a self-amplifying RNA.

A11. The composition or combination of compositions for use as described in any one of embodiments A1 to A10, wherein the vaccine boost composition and the checkpoint inhibitor are each administered separately or on the same day up to 56 days after the vaccine prime composition.

A12. The composition or combination of compositions for use as described in any one of embodiments A1 to A11, wherein the vaccine boost composition and the checkpoint inhibitor are administered on the same day, preferably wherein the vaccine boost composition and the checkpoint inhibitor are administered about 28 days after the vaccine prime composition.

A13. The composition or combination of compositions for use as described in any one of embodiments A1 to A11, wherein the checkpoint inhibitor is administered before the vaccine boost composition, preferably wherein the checkpoint inhibitor is administered about 7 days after the vaccine prime composition and the vaccine boost composition is administered about 28 days after the vaccine prime composition.

A14. The composition or combination of compositions for use as described in any one of embodiments A1 to A11, wherein the checkpoint inhibitor is administered after the vaccine boost composition, preferably wherein the vaccine boost composition is administered about 28 days after the vaccine prime composition and wherein the checkpoint inhibitor is administered about 7 days after the vaccine boost composition, optionally wherein a further dose of vaccine boost composition is administered up to day 84, such as on or around day 84.

A15. The composition or combination of compositions for use as described in any one of embodiments A1 to A11, wherein the checkpoint inhibitor is administered substantially at the same time as the vaccine boost composition, preferably wherein the vaccine boost composition is administered about 28 days after the vaccine prime composition, optionally wherein a further dose of checkpoint inhibitor is administered up to day 84, such as on or around day 84.

A16. The composition or combination of compositions for use as described in any one of embodiments A1 to A15, wherein the vaccine prime composition comprises a viral vectored vaccine.

A17. The composition or combination of compositions for use as described in embodiment A16, wherein the viral vectored vaccine is a simian adenovirus, preferably wherein the viral vectored vaccine is ChAdOx1.

A18. The composition or combination of compositions for use as described in any one of embodiments A1 to A17, wherein the vaccine prime composition comprises a nucleic acid sequence encoding a target antigen against which an immune response is desired, preferably wherein the target antigen is derived from hepatitis B virus (HBV).

A19. The composition or combination of compositions for use as described in embodiment A18, wherein the vaccine prime composition comprises a nucleic acid sequence encoding the same target antigen as the vaccine boost composition.

A20. The composition or combination of compositions for use as described in any one of embodiments A1 to A19, wherein the vaccine prime composition and the vaccine boost composition comprise nucleic acid sequences encoding one or more antigens derived from HBV, optionally including HBV polymerase, HBV core protein and/or HBV surface protein.

A21. The composition or combination of compositions for use as described in any one of embodiments A1 to A20, wherein the subject being treated using the method of treatment suffers from chronic HBV infection (CHB).

A22. The composition or combination of compositions for use as described in embodiment A21, wherein the subject being treated using the method of treatment has undergone antiviral therapy prior to administering the vaccine prime composition, preferably wherein the subject has undergone antiviral therapy for at least 12 months therapy prior to administering the vaccine prime composition.

A23. The composition or combination of compositions for use as described in any one of embodiments A1 to A19, wherein the subject being treated using the method of treatment suffers from cancer.

A24. A method for treating a subject in need thereof, wherein the method comprises administering a vaccine boost composition and a checkpoint inhibitor at least 7 days after administration of a vaccine prime composition.

A25. A kit comprising a vaccine prime composition, a vaccine boost composition and a checkpoint inhibitor as a combined preparation for separate, simultaneous or sequential use in a method of treatment of a viral infection or cancer.

A26. A kit for use according to embodiment A25, wherein the method comprises administering a vaccine boost composition and a checkpoint inhibitor at least 7 days after administration of a vaccine prime composition.

A27. A kit for use according to embodiment A26 for use in the treatment of chronic hepatitis B virus (HBV) infection, optionally wherein the checkpoint inhibitor is an anti-PD-1 antibody, the vaccine prime composition is the viral vectored vaccine ChAdOx1 and the vaccine boost composition is the viral vectored vaccine Modified Vaccinia Virus Ankara (MVA).

A28. A kit comprising a composition or combination according to any of embodiments A1 to A23 for use in a method of treatment, the method comprising administering the vaccine boost composition and the checkpoint inhibitor at least 7 days after administration of the vaccine prime composition.

### EXAMPLES

### Example 1

Administration of a Vaccine Prime Composition Comprising a Non-Replicative Chimpanzee

Adenoviral Vector a T-Cell Response is Generated in Healthy Volunteers and Patients with Chronic Hepatitis B Infection Candidate therapeutic vaccine ChAdOx1-HBV encodes genotype C Hepatitis B (HBV) core, polymerase and surface antigens in a non-replicative chimpanzee adenoviral vector. HBV001 is an open-label, non-randomised, Phase I clinical trial (NCT042979.17) of ChAdOx1-HBV in healthy controls (HC) and patients with chronic HBV (CHB) with supressed HBV DNA on nucleos(t)ide therapy. Participants received low dose (2.5×10⁹ viral particles (vp)) or high dose (2.5×10¹⁰ vp) intramuscular ChAdOx1-HBV. In this example and all subsequent examples unless explicitly stated ChAdOx1-HBV was formulated in isotonic buffered saline comprising of 10 mM L-histidine, 35 mM NaCl, 7.5% sucrose (w/v), 1 mM MgCl2·6H20, 0.1% (w/v) Polysorbate 80, 0.1 mM ethylenediaminetetraacetic acid, 0.5% ethanol (v/v), water for injections to 1 mL, pH 6.6 to a target concentration of 1×10¹¹ vp/mL.

Participants were followed for 168 days for adverse events and HBV serology. HBV specific T cell responses were assessed by interferon-gamma (IFNγ) ELISpot assays using overlapping peptides, 15 amino acids in length, corresponding to the vaccine immunogen (as described in Moore et al (2005) J. Immunology Vol 125, pages 7264-7273).

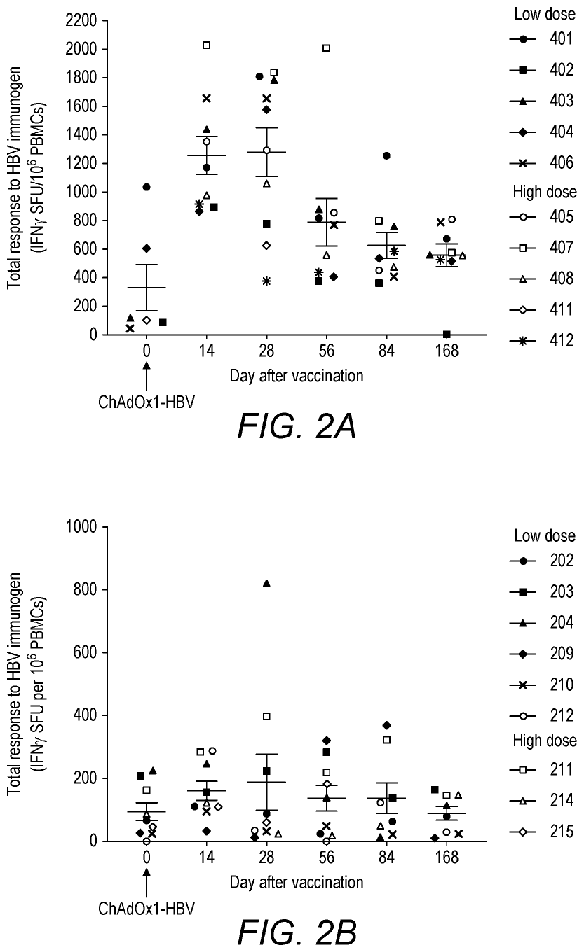

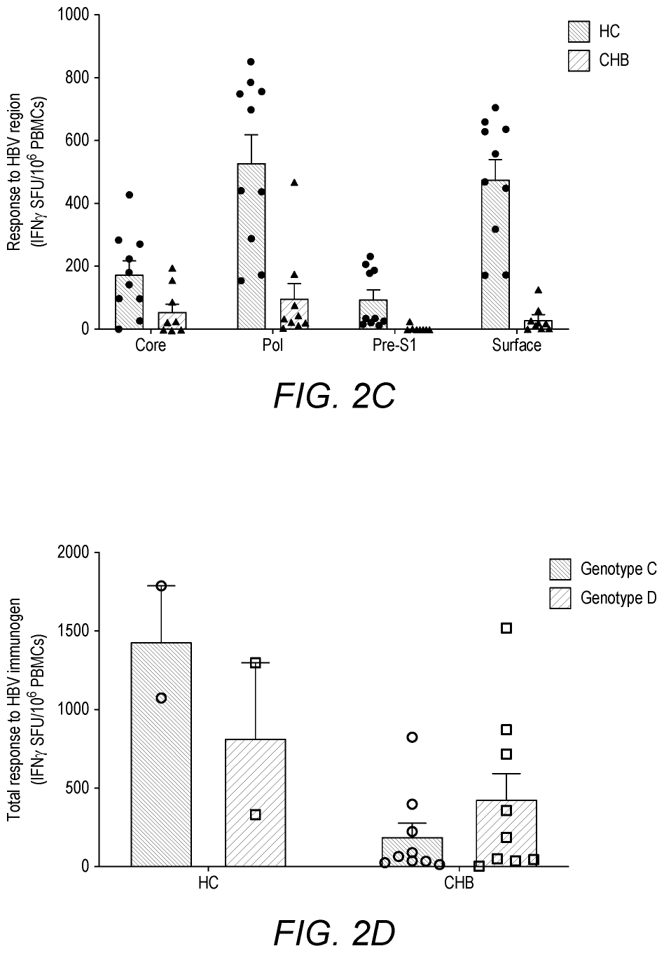

A total of 19 participants were enrolled and received ChAdOx1-HBV at low dose (n=5 HC, n=6 CHB) or high dose (n=5 HC, n=3 CHB). Vaccination was well tolerated with no serious adverse events reported. In HC, injection site pain was the most frequently occurring local adverse event (n=7, 70%) and all cases were mild in severity. In patients with CHB, the only adverse event was an elective laparoscopic procedure, not related to vaccination. Total T cell responses to the HBV immunogen peaked at day 28 post vaccination in both HC (FIG. 2A) and CHB (FIG. 2B). The magnitude of peak T cell responses was significantly higher in HC than in CHB (mean 1284 vs. 189 spot forming units (SFU) per million peripheral blood mononuclear cells (PBMCs), p<0.0001).

The highest magnitude of vaccine induced T cell responses in HC were specific for HBV polymerase (pol) and HBV surface, whereas in CHB the highest responses were specific for HBV pol and HBV core (FIG. 2C). Cross-reactive HBV-specific T cell responses generated by vaccination were reactive to both genotype C and genotype D peptides (FIG. 2D).

### Example 2

Comparison of Immune Response Between Prime Boost Regime Alone and Prime Boost Regime with Administration of a Checkpoint Inhibitor.

A candidate therapeutic HBV vaccine using a chimpanzee adenoviral vector (ChAdOx1-HBV) and a heterologous Modified vaccinia Ankara boost (MVA-HBV), both encoding the inactivated polymerase, core, and the entire S region from a consensus genotype C virus is described in Chinnakannan et al (2020) which is incorporated herein by reference.

A Phase 1b/2a trial has enrolled 64 patients (16 patients each in 4 Groups) with virally-suppressed CHB (on antivirals for a minimum of one year with viral load undetectable and HBsAg <4,000 IU) in Taiwan, South Korea and the UK: Group 1, MVA-HBV (1×10⁸ pfu) followed at d28 by homologous MVA-HBV; Group 2, ChAdOx1-HBV (2.5×10¹⁰ viral particles) followed at d28 by MVA-HBV; Group 3, same as Group 2 with low dose (LD) nivolumab (0.3 mg/kg IV) at d28; Group 4 same as Group 2 with LD nivolumab at d0 and d28 (HBV002, NCT04778904). MVA-HBV was formulated in saccharose 50 g/L, 50 mM NaCl, 10 mM TRIS, 10 mM sodium glutamate, pH 8.0 to a target final concentration of 2.0×10⁸ pfu/mL. Nivolumab was administered via intravenous infusion over 30 minutes, following vaccination by MVA-HBV. The dose used in this study (0.3 mg/kg) is one tenth that commonly indicated for cancer immunotherapy.

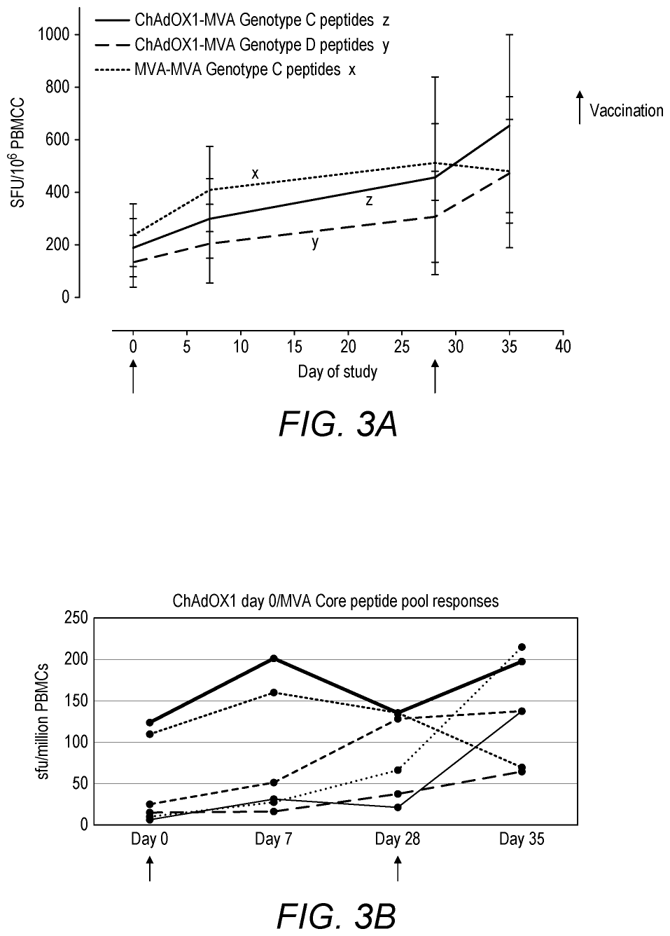

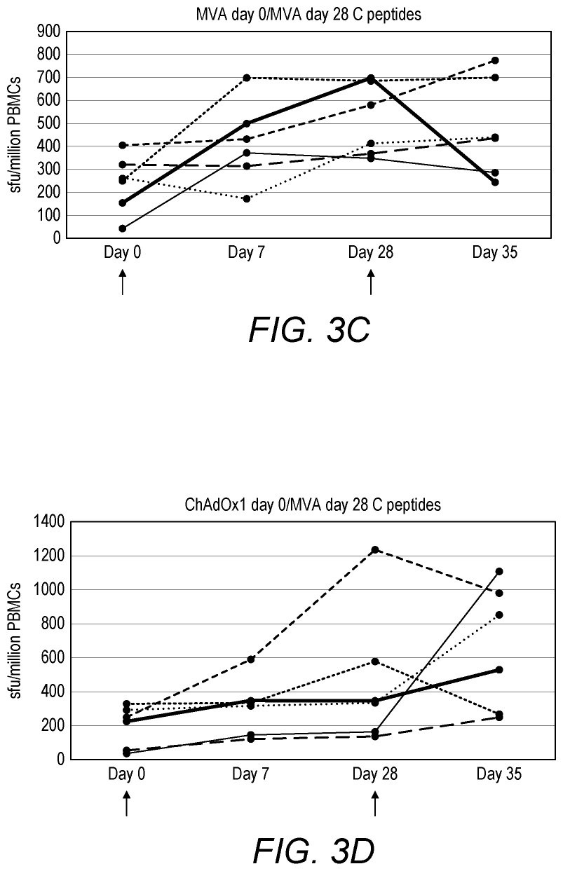

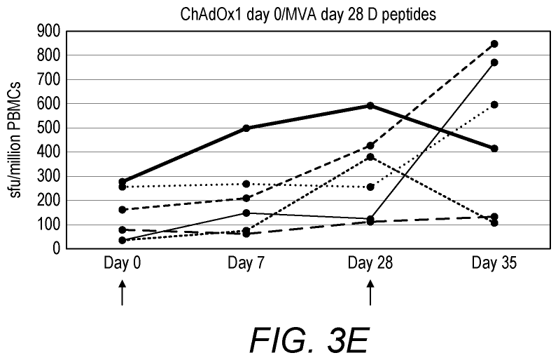

The results are provided for the first six patients in Groups 1 and 2, all from Taiwan sites, who had reached a day 35 time point for immunogenicity assessment in September (FIG. 3). The enrolment criteria for the study include being on effective treatment for HBV for one year, levels of HBV DNA <40 copies/ml, surface antigen (sAg)<4000 IU. Initial results use a qualified Gamma interferon ELISpot assay. Immunogenicity was monitored for the first 6 patients in Groups 1 and 2 through 35 days, the likely peak of the immune response. Cryopreserved PBMCs were stimulated using 7 HBV peptide pools representing PreS1+PreS2, Core, 4 separate pools of pol and surface antigen (sAg) from the vaccine sequence or from a consensus genotype D, along with positive and negative controls. The total response (DMSO subtracted) is shown, as well as the core-specific response. Pol, as the largest component of the vaccine, dominated. The best response was achieved following a heterologous prime-boost regimen (ChAdOx1-HBV followed by MVA-HBV). There is good cross-reactivity to D-specific peptides. Administration of the therapeutic vaccine induces antigen specific T cell responses to all antigens, with robust responses to core and polymerase, as compared to healthy controls, who exhibit a greater response to surface antigen.

The vaccine regimens were well-tolerated in CHB patients and induced T cells were shown to be cross-reactive to two major HBV genotypes (genotype C and genotype D).

### Example 3

Administration of a Checkpoint Inhibitor with a Therapeutic HBV Vaccine Increases Clearance of HBV.

64 participants were enrolled who were chronically infected with Hepatitis B infection and virally suppressed with approved oral anti-HBV therapies (HBV002, NCT04778904) as described for Example 2. A study was conducted to compare the immunogenicity of MVA-HBV alone (Group 1), ChAdOx1-HBV followed by MVA HBV (Group 2), ChAdOx1-HBV followed by MVA HBV with nivolumab at day 28 (Group 3) and ChAdOx1-HBV followed by MVA HBV with nivolumab at day 0 and day 28 (Group 4). ChAdOx1-HBV was administered at 2.5×10¹⁰ virus particles per dose. MVA-HBV was administered at 1×10⁸ plaque forming units (pfu) per dose. Nivolumab was administered at 0.3 mg/kg. All treatment groups received study vaccine on Day 0 and Day 28.

ChAdOx1-HBV is a genetically-modified (GM) non-replicating chimpanzee adenovirus vector encoding HBV consensus sequences from a group C genotype. The ChAdOx1 virus has been engineered to be replication deficient. ChAdOx1-HBV is formulated in isotonic buffered saline comprising of 10 mM L-histidine, 35 mM NaCl, 7.5% sucrose (w/v), 1 mM MgCl2·6H2O, 0.1% (w/v) Polysorbate 80, 0.1 mM ethylenediaminetetraacetic acid, 0.5% ethanol (v/v), water for injections to 1 mL, pH 6.6 to a target concentration of 1×10¹¹ vp/mL.

MVA-HBV is a GM poxvirus that is non-replicating in mammalian cells encoding the same HBV consensus sequences as ChAdOx1-HBV. The MVA virus is no longer able to replicate in humans and has safely been administered to over 130,000 people. It both boosts and prolongs the CD4+ and CD8+ T cells induced by ChAdOx1. MVA-HBV is formulated in saccharose 50 g/L, 50 mM NaCl, 10 mM TRIS, 10 mM sodium glutamate, pH 8.0 to a target final concentration of 2.0×10⁸ pfu/mL.

HBV disease markers in serum were analysed at screening, pre-vaccination on Days 0 and 28, on Days 7 and 35, and on Month 3. HBV infection markers monitored included (HBsAg, Hepatitis B surface antibody [HBsAb] seroconversion, hepatitis B DNA, HBeAg).

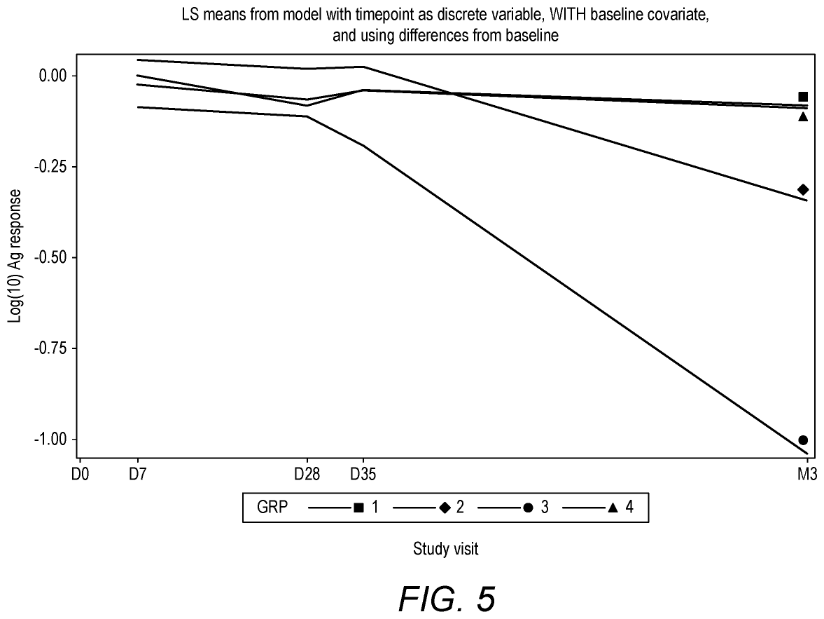

Table 1 summarizes the data obtained for Hepatitis B surface antigen (HBsAg) level up to 3 months. The mean level at day 0 is provided, as well as the log₁₀ decline at month 3. FIG. 5 is a plot of the least squared means for each group at the different timepoints, determined using a model with timepoint as a discrete variable, with the baseline covariate, and using differences from the baseline.

The checkpoint inhibitor treatment dose in groups 3 and 4 is significantly lower than the dose approved for treatment.

TABLE 1

| | Mean D 0 | Log decline |
|---|---|---|
| Dosage Regime | SAg level | at month 3 |
| MVA D 0, MVA D 28 (N = 9) (Group 1) | 747 | −0.09 |
| ChAdOx1 D 0, MVA D 28 (N = 6) (Group 2) | 874 | −0.36 |
| ChAdOx1 D 0, MVA + nivolumab D 28 (N = | 746 | −1.04 |
| 6) (Group 3) | | |
| ChAdOx1 + nivolumab D 0, MVA + | 1111 | −0.12 |
| nivolumab D 28 (N = 5) (Group 4) | | |

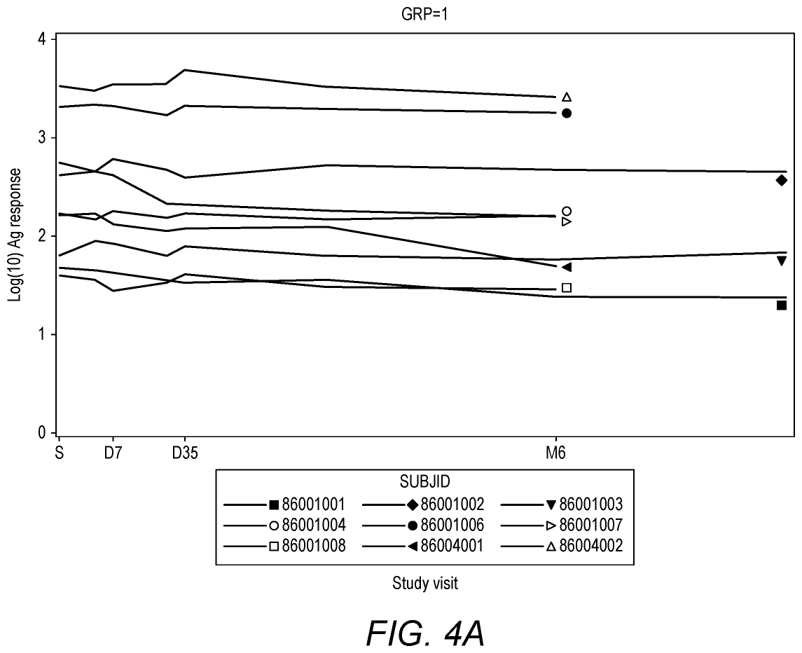

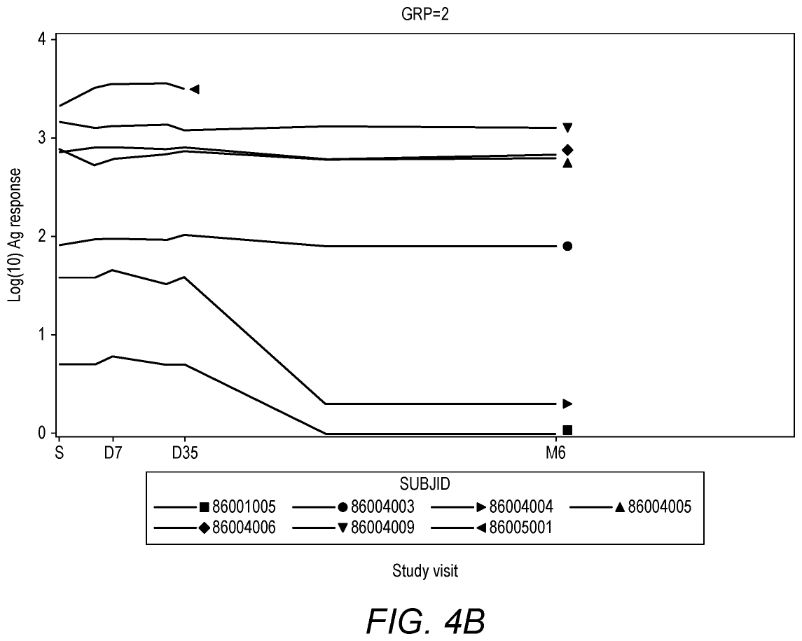

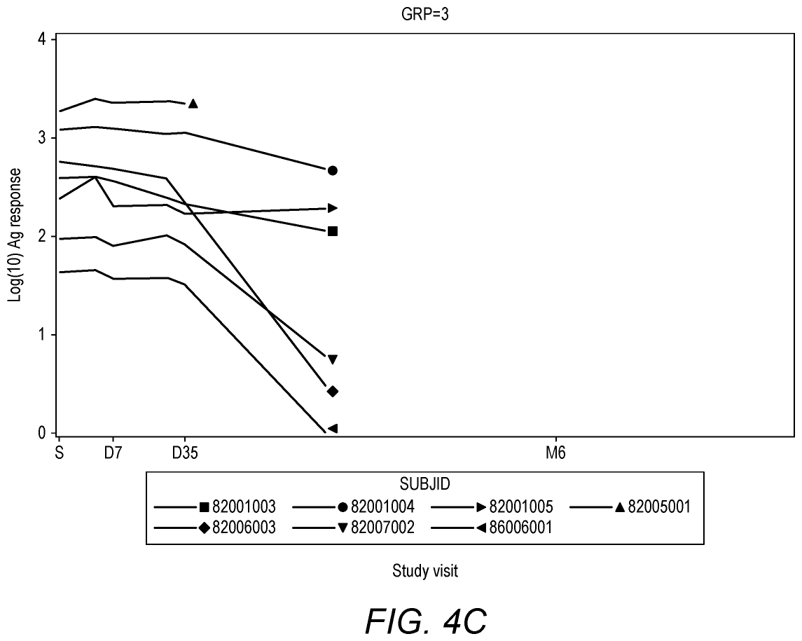

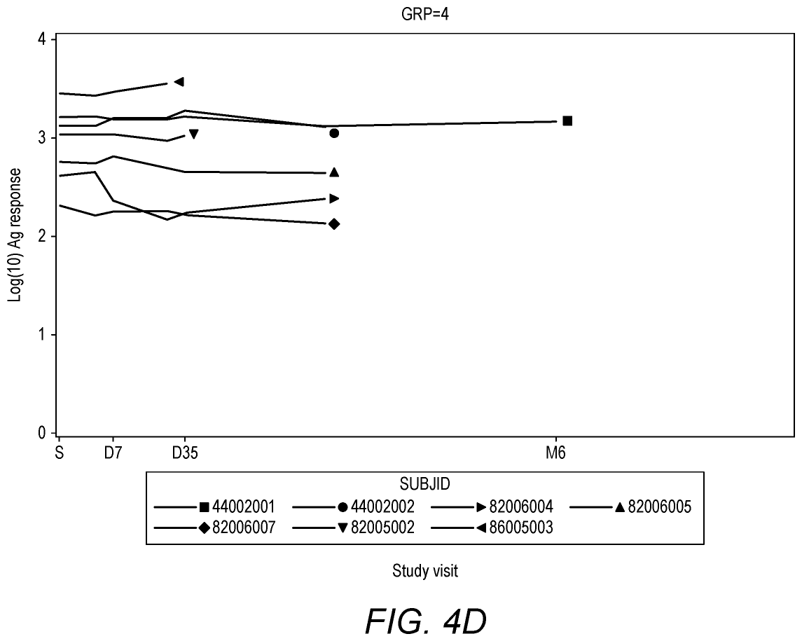

The Hepatitis B surface antigen level for the patients in Group 3 are given in Table 2. Prior art techniques provide an expected drop at 1 year for the population to be 0.1 log. In group 3 all patients achieved greater than 0.3 log drop in three months, and 3/6 were greater than 1 log. One patient no longer had detectable surface antigen. The p-value of Group 3 versus group 1 is p=0.01. FIG. 4 shows the Log(10) response for each patient in each group (4A=group 1; 4B=group 2, 4C=group 3, 4D=group 4) as a function of time.

Data from the 27 patients, who had completed 3 months in the HBV002 study in chronic Hepatitis B (CHB) patients, demonstrated noted changes in surface antigen (HBsAg) levels. Surprisingly, the greatest changes in surface antigen levels were seen in the group receiving low-dose nivolumab with the heterologous boost.

TABLE 2

| Patient | Mean D 0 SAg level | Log decline at month 3 |
|---|---|---|
| 1 | 399 | −0.31 |
| 2 | 398 | −0.43 |
| 3 | 1289 | −0.41 |
| 4 | 514 | −2.26 |
| 5 | 45 | Not detected |
| 6 | 98 | −1.20 |

Analysis of Group 3, patients on antivirals for at least 12 months with undetectable HBV DNA and a mean starting HBsAg level of 441 IU/ml, showed: Mean of greater than one log decrease (−1.04) and a greater than one log decrease in HBsAg in 3/6 patients at 3 months; HBsAg in one patient was undetectable 3 months after starting the immunotherapy regimen; One patient with a decrease experienced a transaminase flare after the MVA boost plus nivolumab that resolved over 3 weeks; Despite the very small patient numbers the difference in mean HBsAg between Group 3 and the other groups was highly significant (p<0.01).

The analysis shows that some patients on chronic antivirals receiving VTP-300 alone (i.e. a prime boost regimen including ChAdOx1 and MVA, e.g. group 2), as well as in combination with a low-dose checkpoint inhibitor, experienced meaningful decreases in HBsAg levels. HBsAg is a hallmark of chronic HBV infection. Fewer than 10% of patients on current standard of care HBV therapies ever achieve distinct, sustained HBsAg decrease or loss, a state associated with functional cure of the disease.

### Example 4

A Phase 1b/2a trial enrolled 55 patients with virally-suppressed CHB (on antivirals for a minimum of one year with viral load undetectable and HBsAg <4,000 IU/mL) in Taiwan, South Korea and the UK (NCT047789) (Table 3). The study was a continuation of trial NCT04778904 previously described therefore the design was as described in the previous examples. Briefly, Group 1, received MVA-HBV (1×10⁸ pfu) followed at d28 by homologous MVA-HBV (homologous prime-boost regimen); Group 2, received ChAdOx1-HBV (2.5×10¹⁰ viral particles) followed at d28 by MVA-HBV (heterologous prime-boost regimen); Group 3, received the same as Group 2 with low dose (LD) nivolumab (0.3 mg/kg IV) at d28 (heterologous prime-boost regimen with checkpoint inhibitor administered on same day as boost); Group 4 received the same as Group 2 with LD nivolumab at d0 and d28 (heterologous prime-boost with checkpoint inhibitor administered twice, on same day as prime and boost)(NCT04778904). Individual plots show results for those patients with data through to at least the end of month 3.

Hepatis B specific efficacy and immunologic parameters were followed. An amendment to the study closed Groups 1 and 4 early due to interim HBsAg data. Visits were conducted at days 0, 7, 28, 35 and months 3, 6 and 9. Samples including blood and urine samples were taken at each time point. Peripheral blood mononuclear cells (PBMCs) were used for immunological testing. Serum blood samples were used for analysis of HBsAg.

TABLE 3

| | | Gender | Total | Through | Through | Through | Through |
|---|---|---|---|---|---|---|---|
| Group | Age (Yrs) | (M:F) | participants | D 35 | Mo 3 | Mo 6 | Mo 9 |
| 1 | 52.6 +/− 6.8 | 8:1 | 10 (9)* | 9 | 9 | 9 | 9 |
| 2 | 53.3 +/− 6.7 | 15:3 | 18 | 13 | 10 | 8 | 6 |
| 3 | 49.8 +/− 8.3 | 11:7 | 18 | 14 | 11 | 8 | 6 |
| 4 | 49.9 +/− 15.4 | 7:2 | 9 | 9 | 9 | 7 | 5 |

Hepatitis B Surface Antigen Response

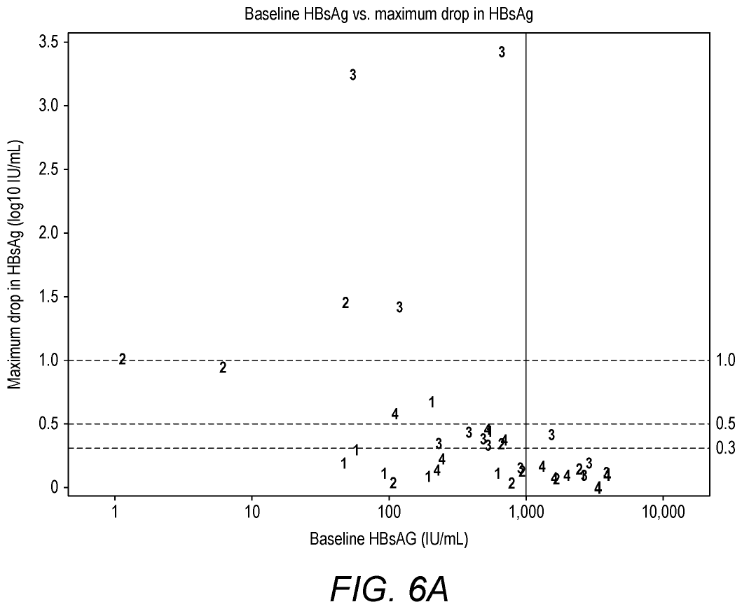

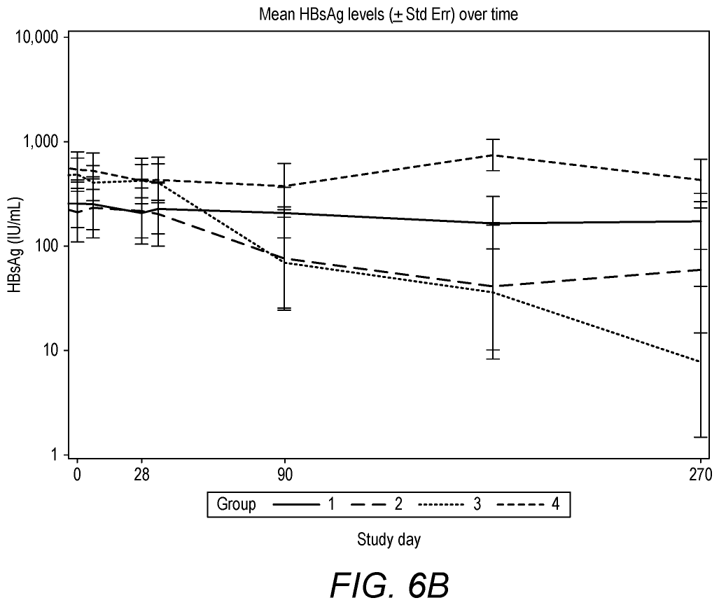

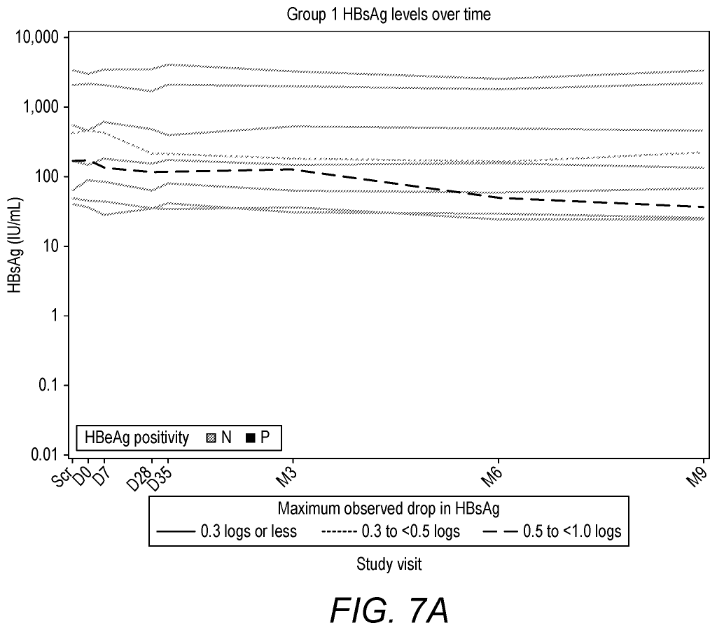

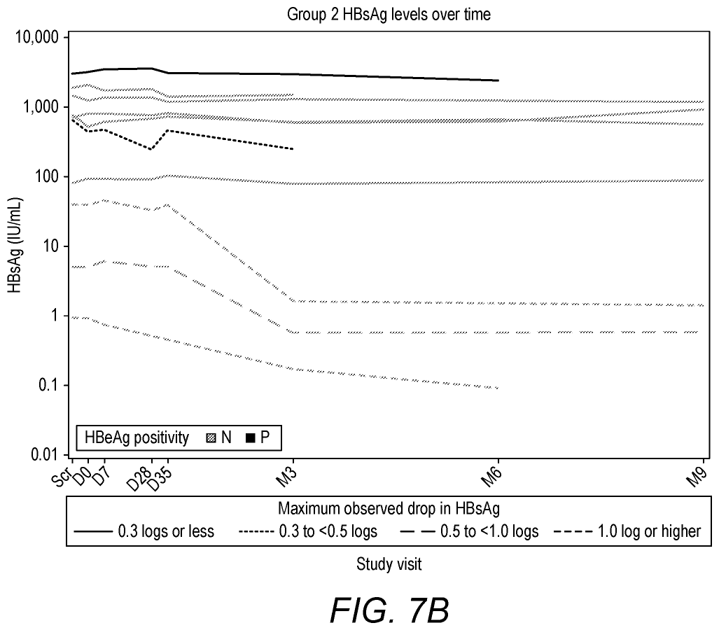

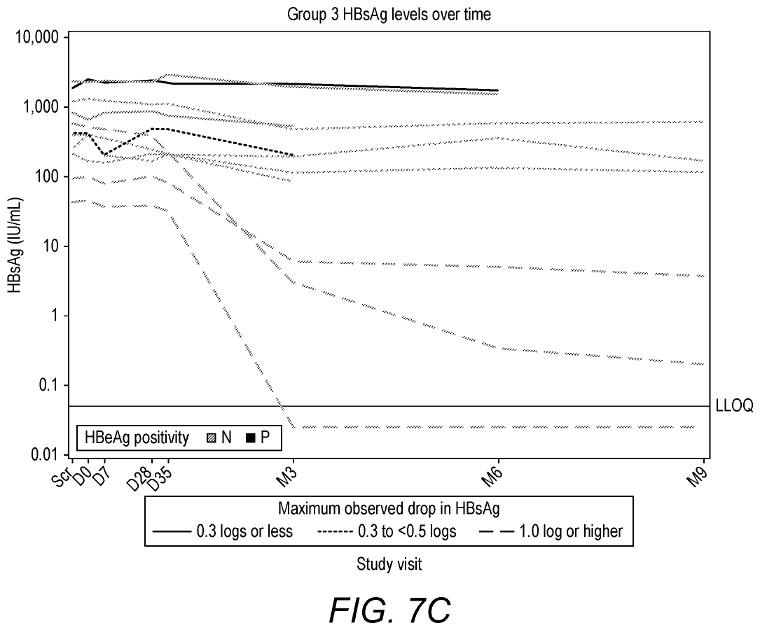

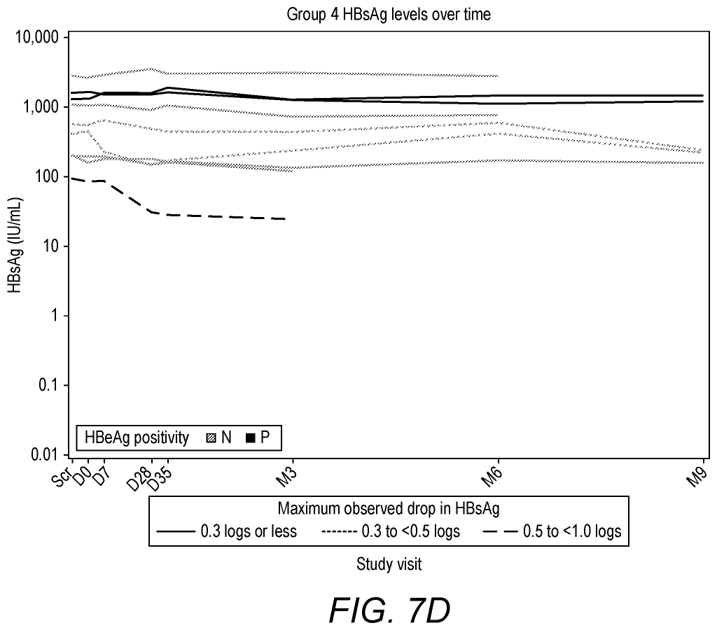

HBsAg data shown were collected through May 9, 2022. FIG. 6 shows the HBV surface antigen responses by group, and FIG. 7 shows the surface antigen responses by individual. Individual plots show results for those with visits through months 3, 6 and 9.

Significant, durable reductions of HBsAg were seen in patients in the Group 2 (heterologous prime-boost regimen): 3 patients had 0.7, 0.7 and 1.4 log₁₀ declines 2 months post last dose. These dramatic declines persisted in all 3 patients at latest follow-up 5 or 8 months after the last dose (the boost dose). These 3 patients had baseline HBsAg under 50 IU/mL. Patients with low baseline HBsAg (e.g. <50 IU/mL) responded better in this study. For the first 8 patients in Group 3 who received a single low dose of nivolumab at the time of the booster dose, the mean reduction in HBsAg was greater than 1 log₁₀ at 6 months. This effect persisted with a mean decline of 1.15 log₁₀ at 8 months after the last dose In this group the effect was most prominent for patients with starting values under 1,000 IU/mL. One patient in group 3 developed a non-detectable HBsAg level, which continued 8 months after last dose. The lowering of HBsAg persisted until the last measurement in all patients with >0.5 log₁₀ reduction. This is an improvement over direct acting agents to date where the response is much less durable (e.g. expected drop at 1 year for the population <0.1 log). No reductions ≥1 log₁₀ were seen in Group 1 (homologous prime-boost) patients who received 2 doses of MVA-HBV, or in patients who received low-dose nivolumab with both doses in the heterologous prime-boost regimen (Group 4). These groups were discontinued after the interim analysis described in Example 3.

The antigen in the trials included modified (inactivated) polymerase (Pol), core, Pre-S1 and Pre-S2 and S polypeptides with a consensus Genotype C sequence (SEQ ID NO: 8 and SEQ ID NO: 11-16). Immunologic assays were performed with peptide pools encompassing core, Pol (4 pools) pre-S1, pre-S2, S for the gamma IFN ELISpot assays (performed as for Example 1) and 4 pools (Core, Pol1, Pol2, S) for the intracellular cytokine staining (ICS) assay. All assays were short, 6-hour to overnight stimulations without in vitro expansion. The ICS used the following phenotypic and activation markers: CD3, CD4, CD8, IFNγ, IL-2, TNF-alpha, CCR7; CD45RO; CD107; CD154.

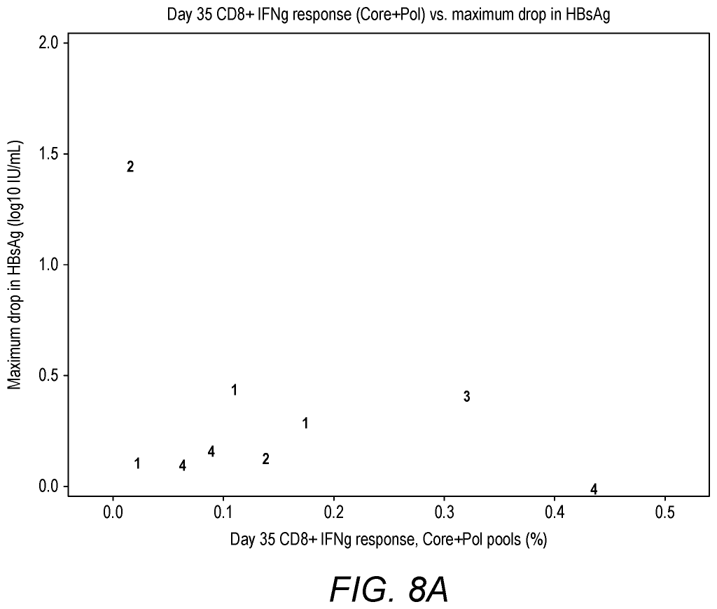

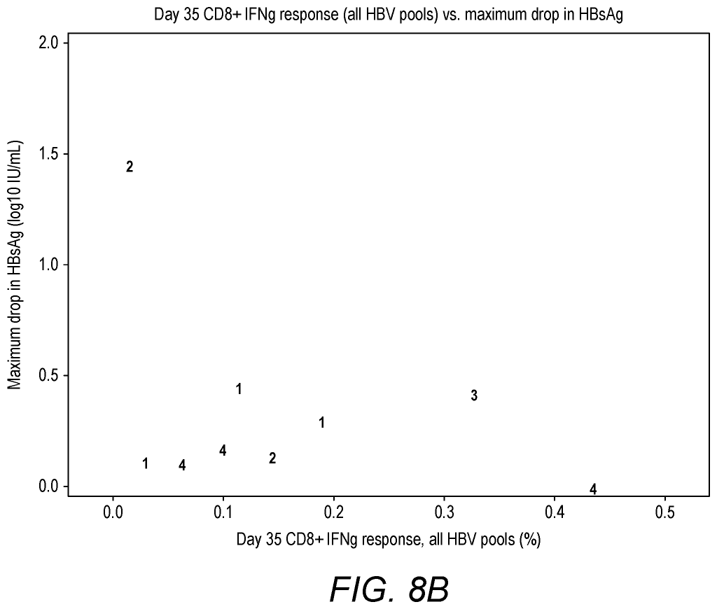

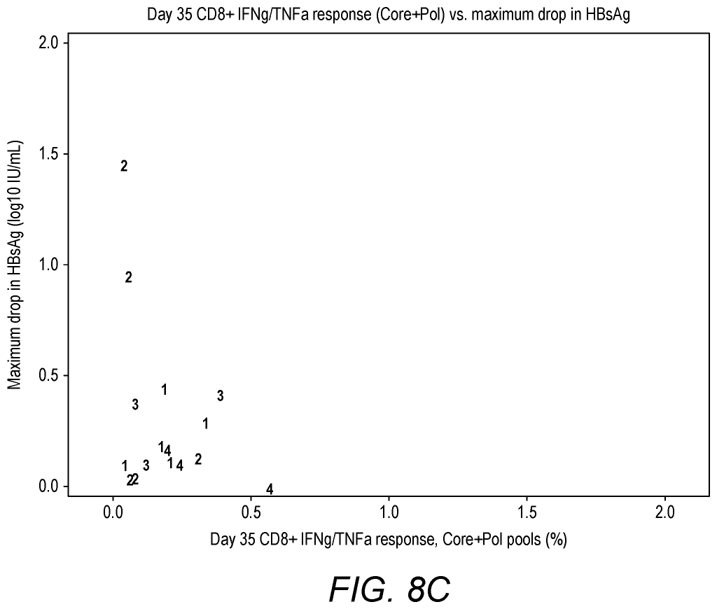

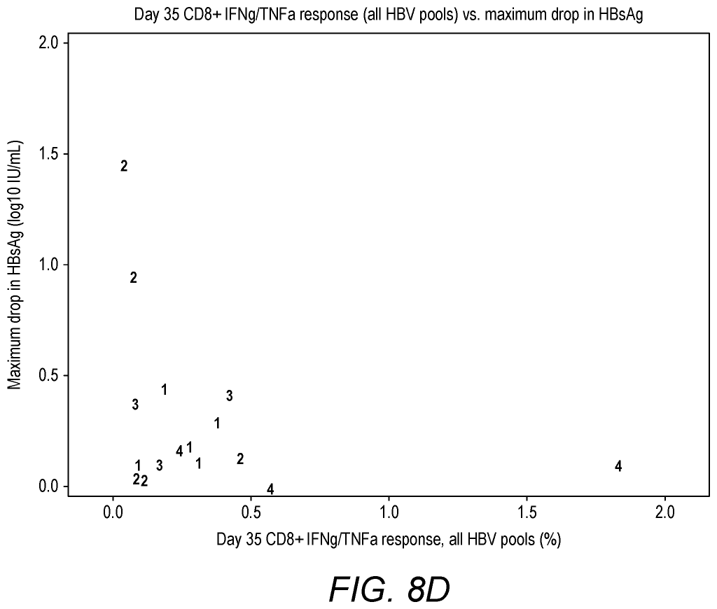

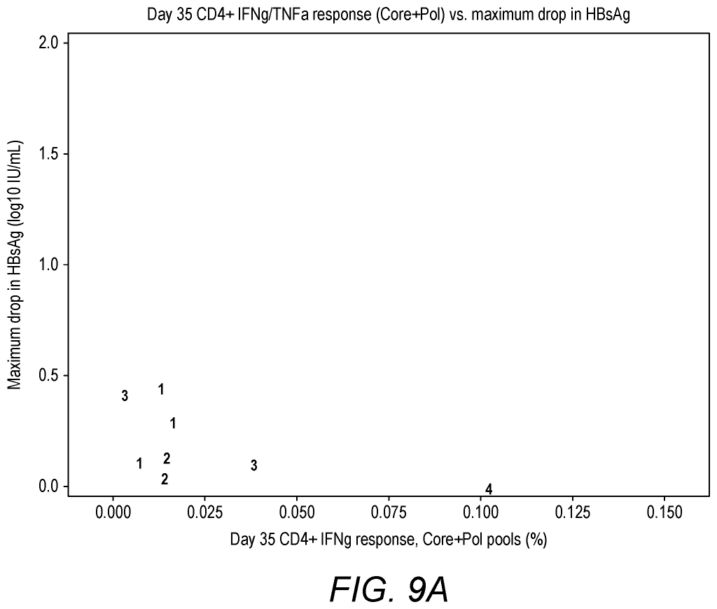

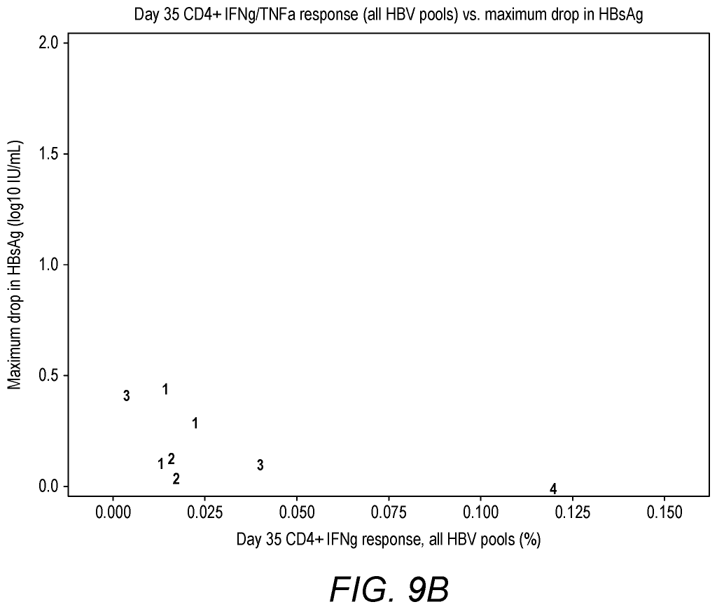

FIGS. 8 to 11 show the polyfunctional T-cell response at the peak time point, day 35 (following the prime at day 0 and boost at day 28 as described above). For FIGS. 8 to 11 the maximum drop in HBsAg is plotted on the Y-axis with units Log 10 IU/mL. FIG. 8 panel A and B show the CD8+ T-cell interferon gamma (IFNγ) response (single cytokine response) and panels C and D show the CD8+ T-cell IFNγ and TNFa response (dual cytokine response) vs. maximum drop in HBsAg for (Core+Pol) and all HBV pools. FIG. 9 shows the CD4+ T-cell interferon gamma (IFNγ) response (single cytokine response) vs. maximum drop in HBsAg for Core+Pol (panel A) and all HBV pools (panel). The Y-axis is on the same scale as FIG. 8 for ease of comparison.

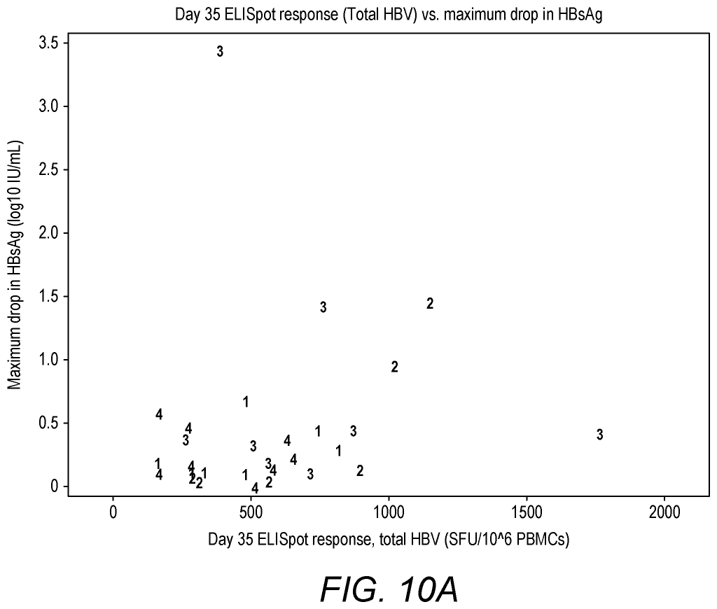

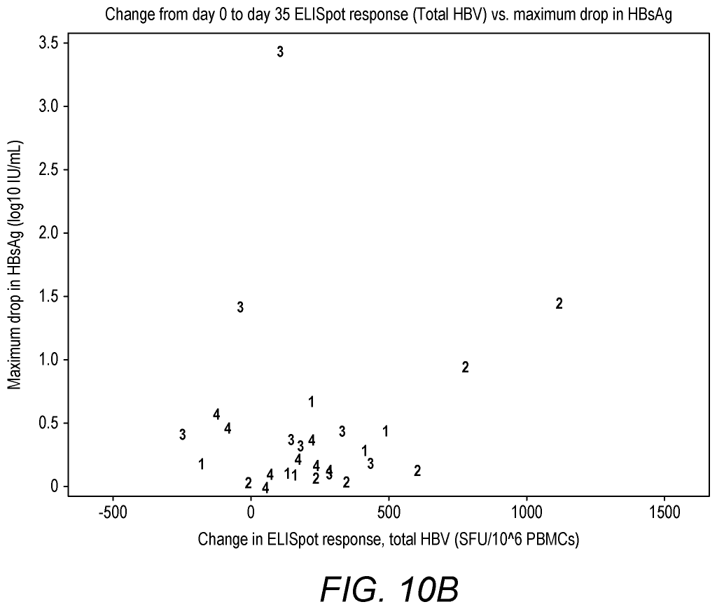

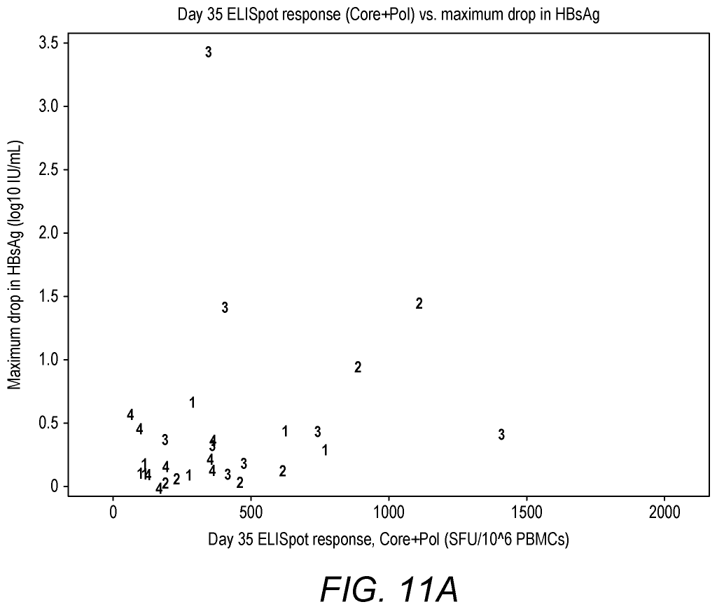

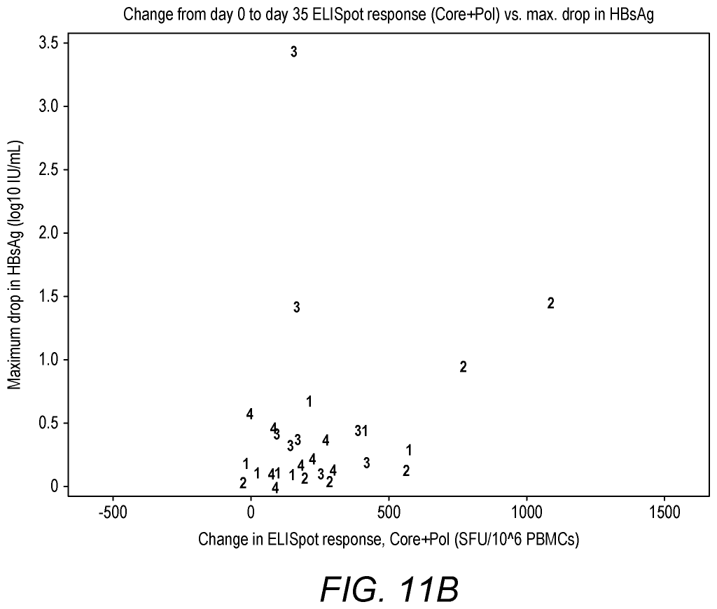

FIGS. 10 and 11 provides the Day 35 ELISPOT response for Total HBV (all combined peptide pools)(FIG. 10) and Core+Pol combined peptide pools (FIG. 11) vs. maximum drop in HBsAg, and the change in ELISpot response from DO to Day 35.

IFNγ responses to peptide pools derived from HBV antigens were measured in PBMCs using ELISpot assays.

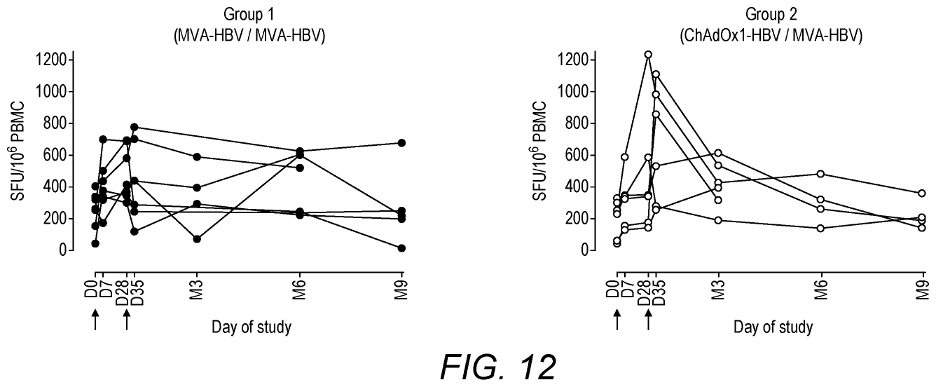

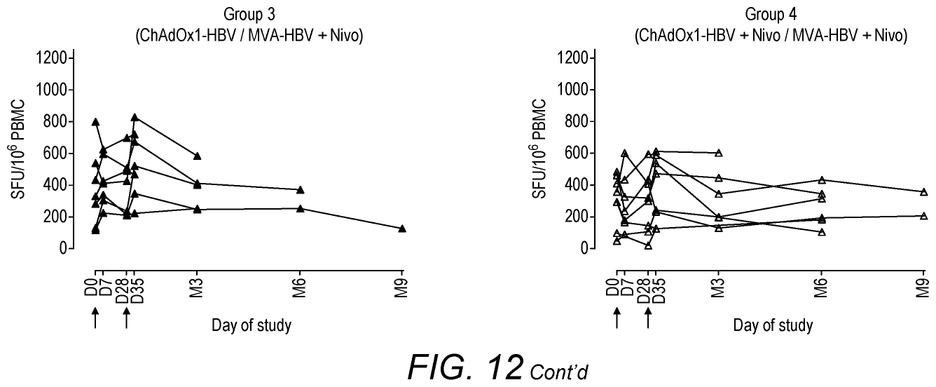

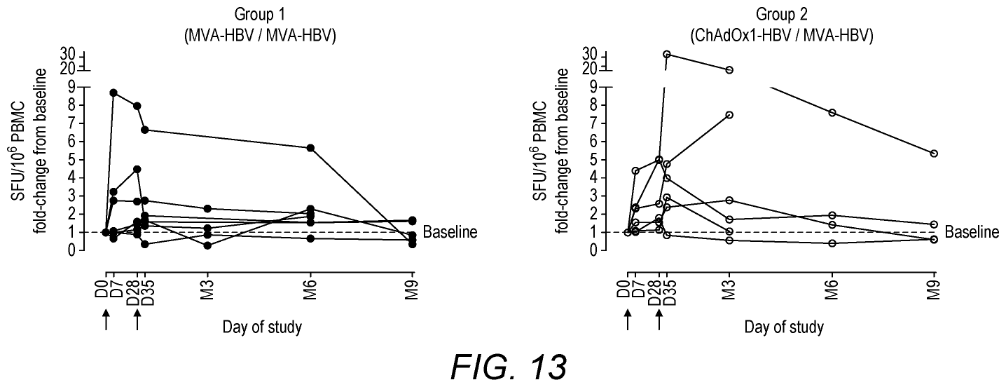

FIG. 12 provides the IFNγ ELISPOT data for the sum of responses to peptide pools derived from all HBV antigens (Core+Pol+Pre-S+S) in each of groups 1-4 (Y axis: SFU/10⁶ PBMC).

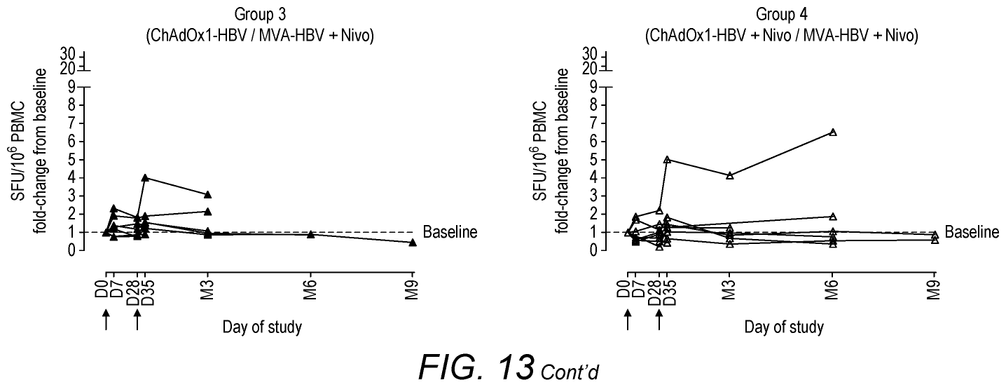

FIG. 13 provides the IFNγ ELISPOT data for the sum of responses to HBV antigens (Core+Pol+Pre-S+S), plotted as fold change from baseline in each of groups 1-4 (Y axis: SFU/10⁶ PBMC fold change from baseline).

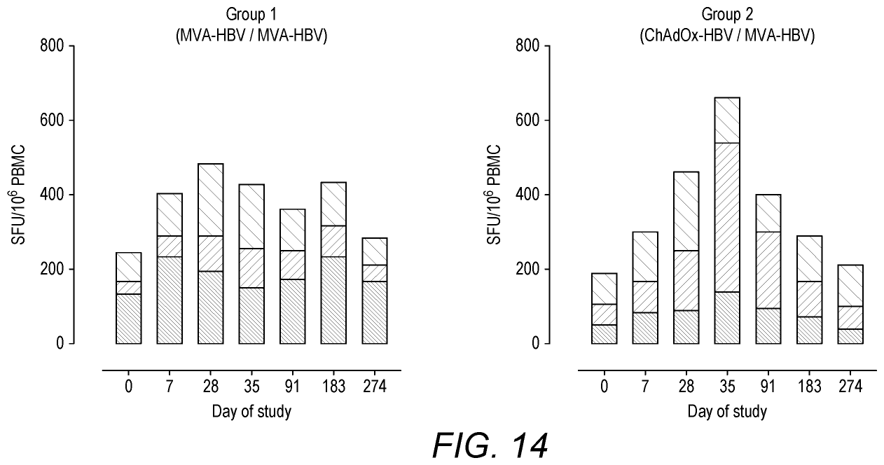

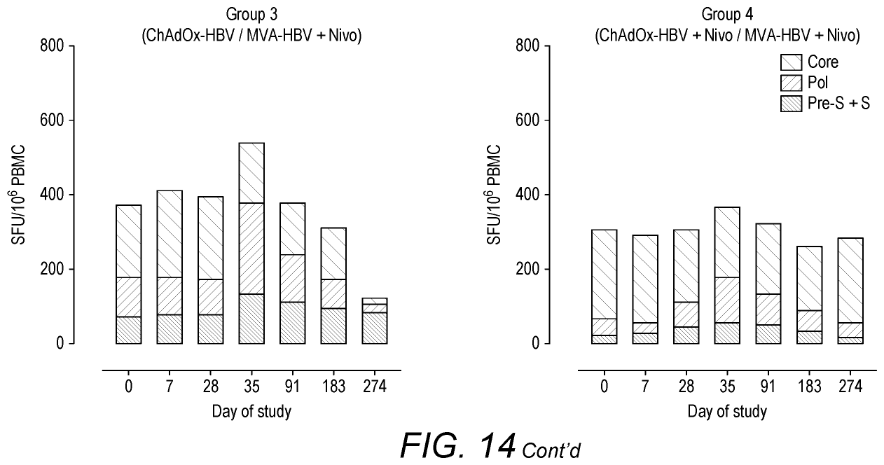

FIG. 14 provides the ELISpot data as stacked bars representing the responses to HBV antigens (Core+Pol+Pre-S+S) in each of groups 1-4 (Y axis: SFU/10⁶ PBMC fold). Response to core is shown in black, to Pol in dark grey and to Pre-S+S in light grey.

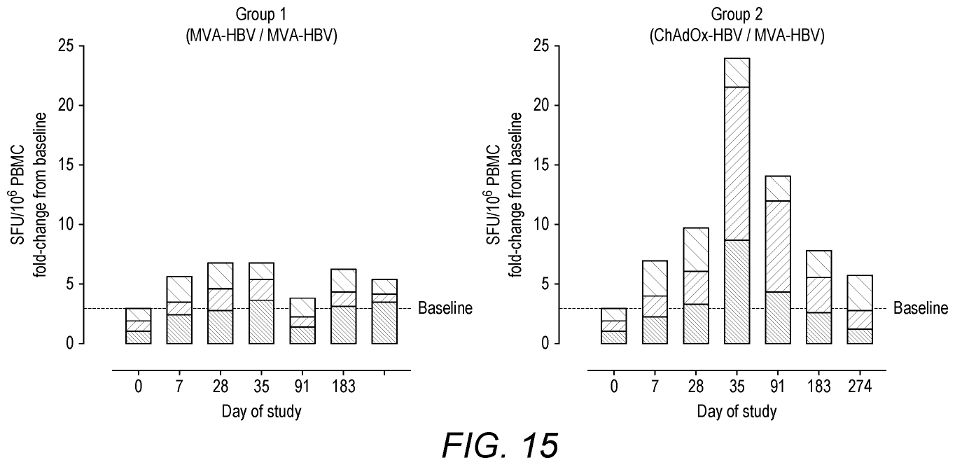

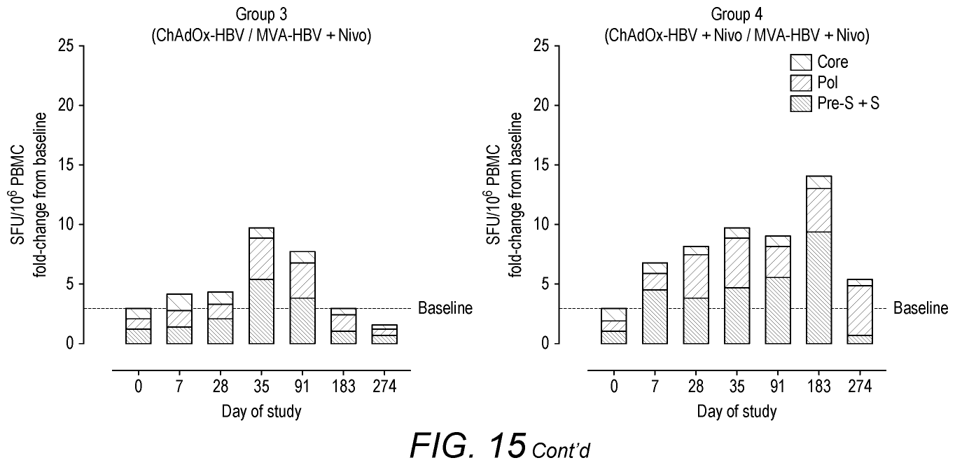

FIG. 15 provides the IFNγ ELISPOT data for the sum of responses to HBV antigens (Core+Pol+Pre-S+S), fold change from baseline in each of groups 1-4.

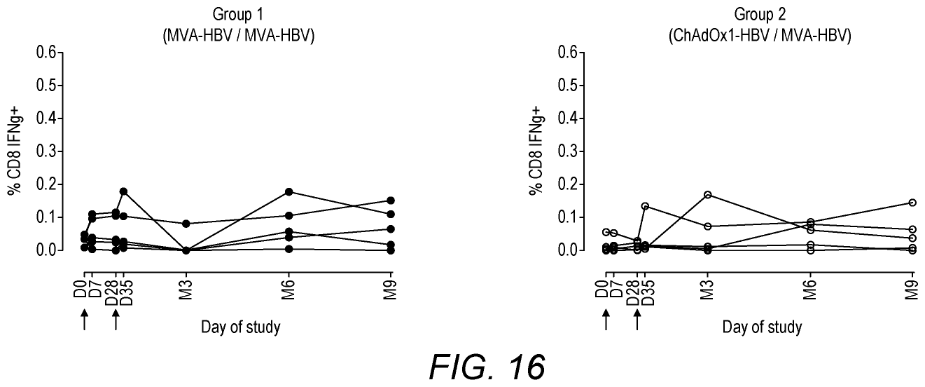

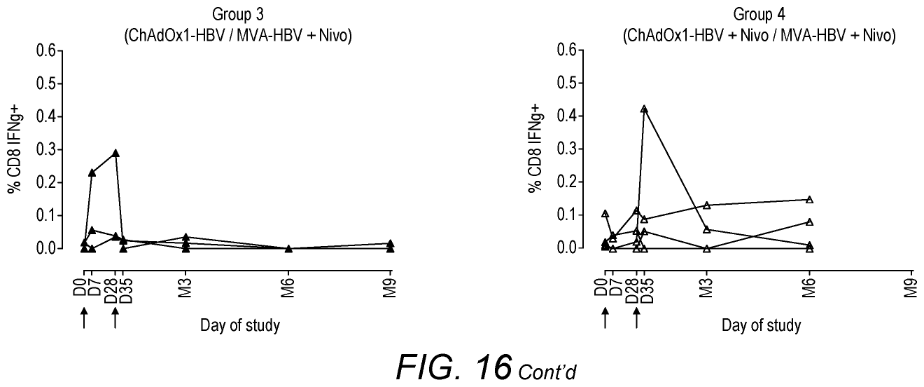

FIG. 16 provides the ICS data for the sum of CD8+ IFNγ responses to HBV antigens (Core/Pol1/Pol2, Pol3/Pol4, Pre-S1-2+S) in each of groups 1-4.

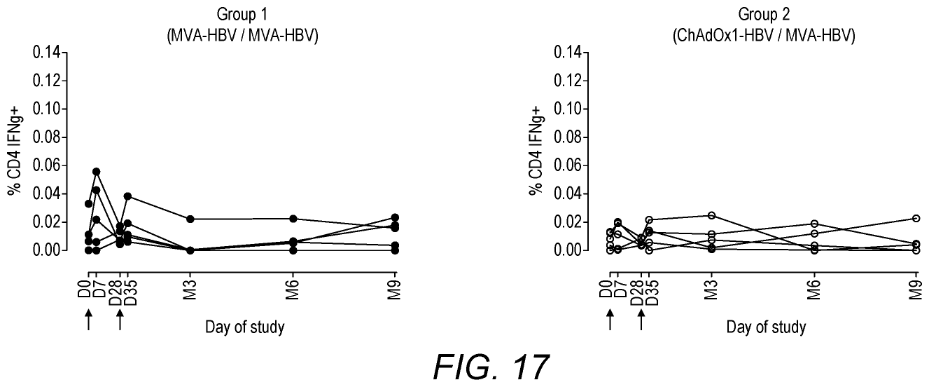

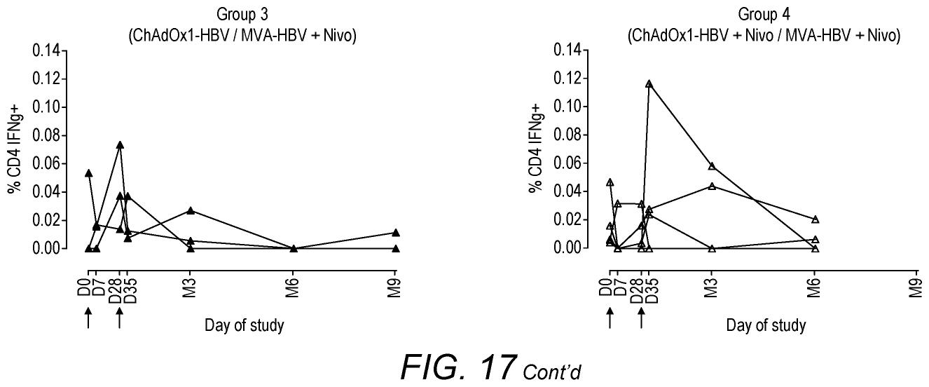

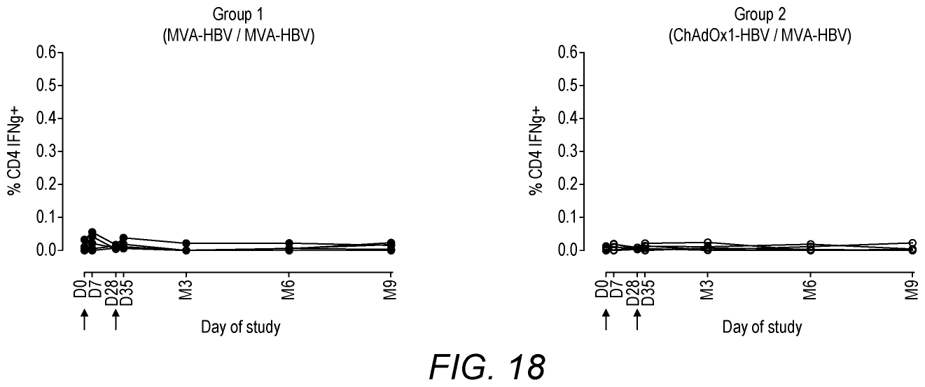

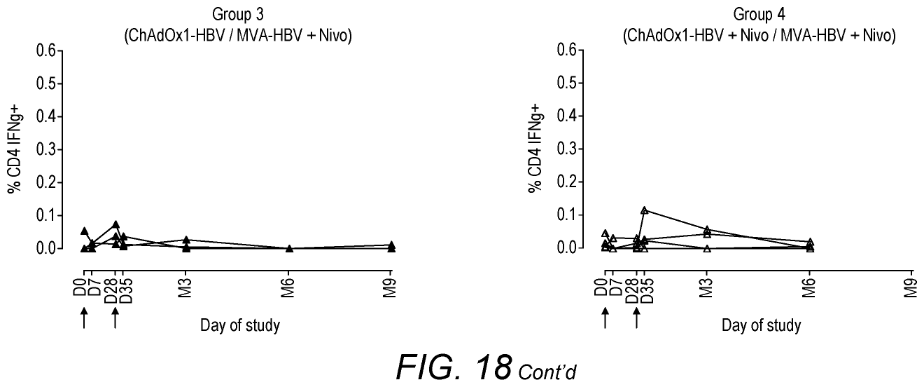

FIG. 17 provides the ICS data for the sum of CD4+ IFNγ responses to HBV antigens (Core/Pol1/Pol2, Pol3/Pol4, Pre-S1-2+S) in each of groups 1-4. FIG. 18 provides the same data but with the Y axis on the same scale as FIG. 16 for ease of comparison to the CD8+ IFNγ response.

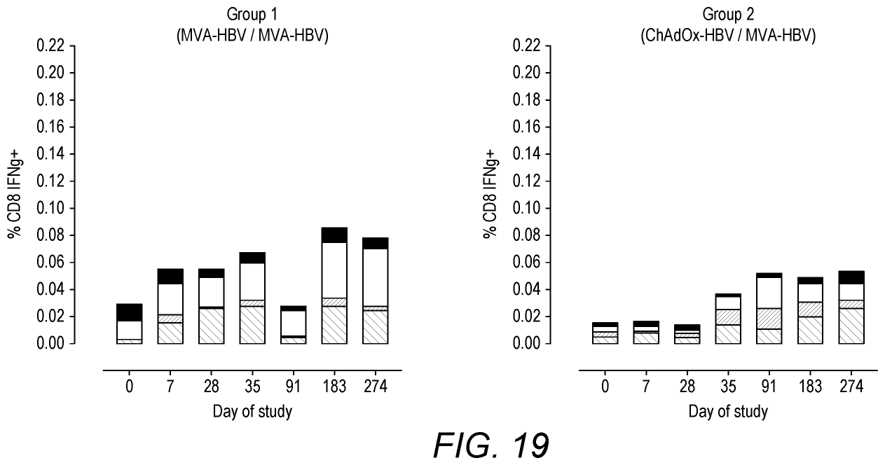

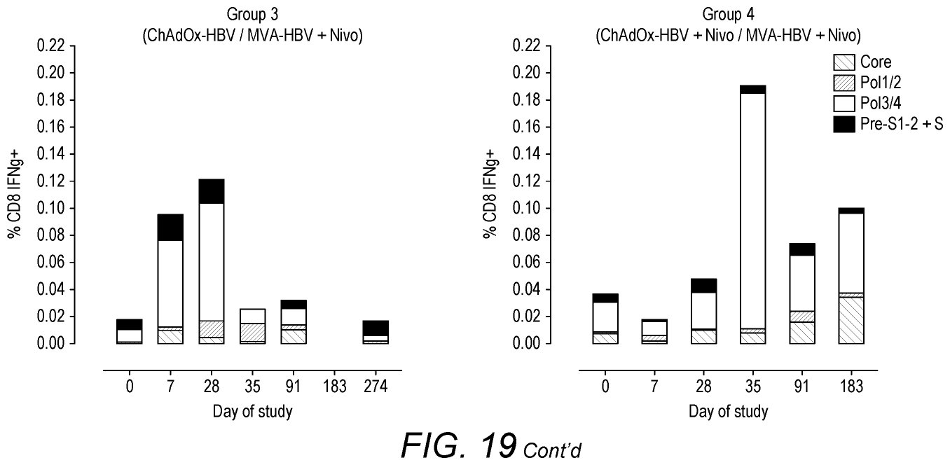

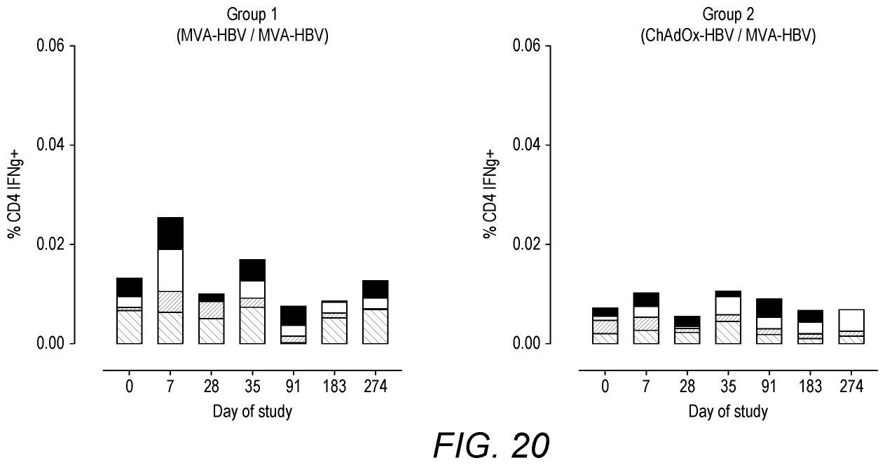

FIG. 19 provides the CD8 IFNγ data as stacked bars representing the mean response to HBV antigens (Core+Pol1/2, Pol3/4, Pre-S1-2+S) in each of groups 1-4 (Y axis: % CD8 IFNγ+).

Response to core is shown in dark grey, to Pol1/2 in light grey, to Pol3/4 in white and to Pre-S1-2+S in black.

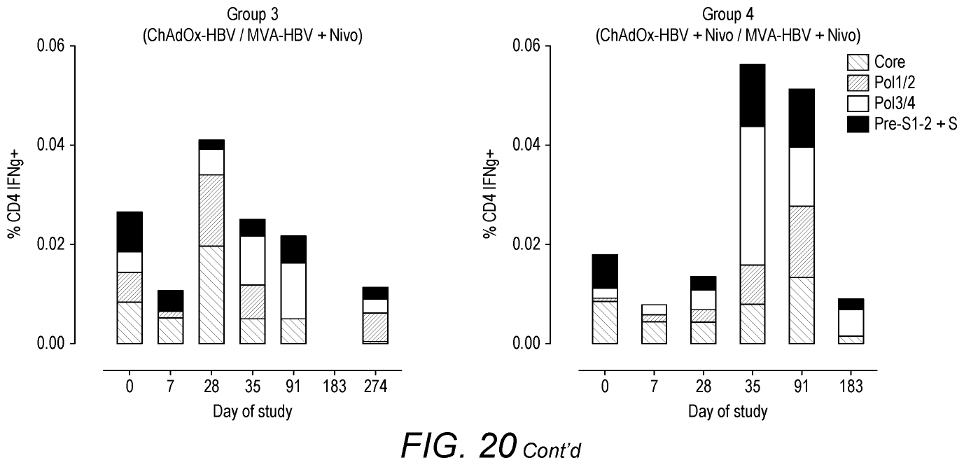

FIG. 20 provides the CD4 IFNγ data as stacked bars representing the mean response to HBV antigens (Core+Pol1/2, Pol3/4, Pre-S1-2+S) in each of groups 1-4 (Y axis: % CD4 IFNγ+).

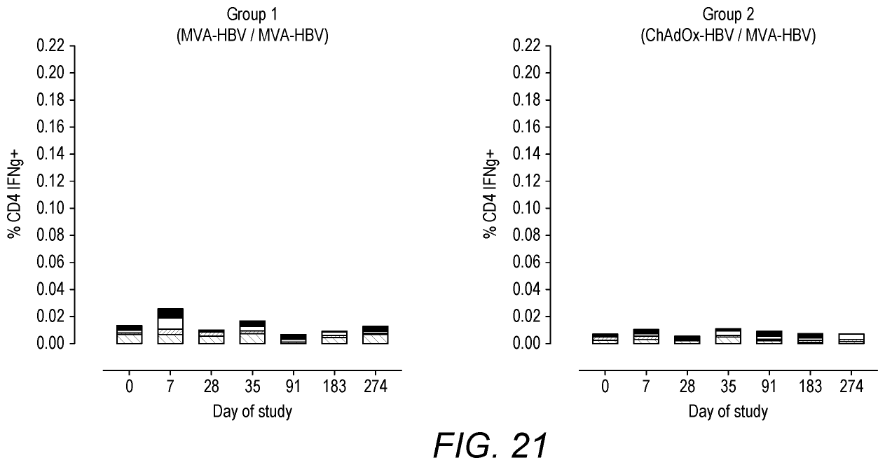

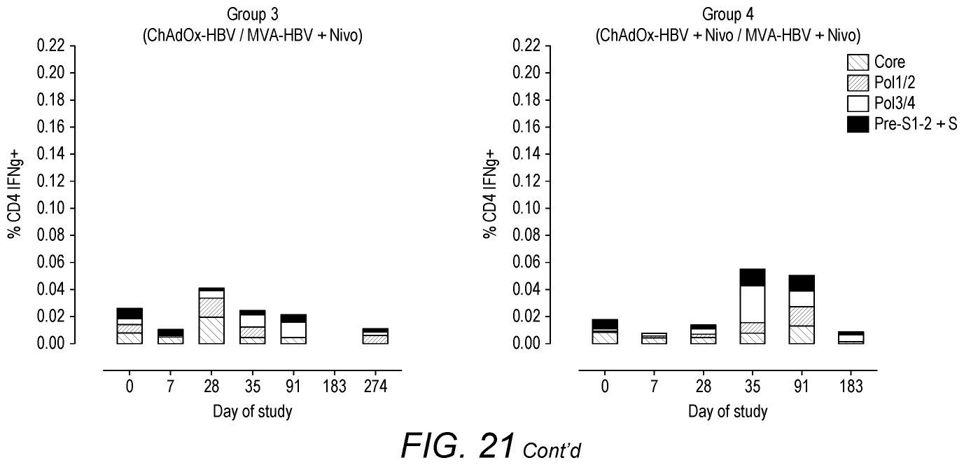

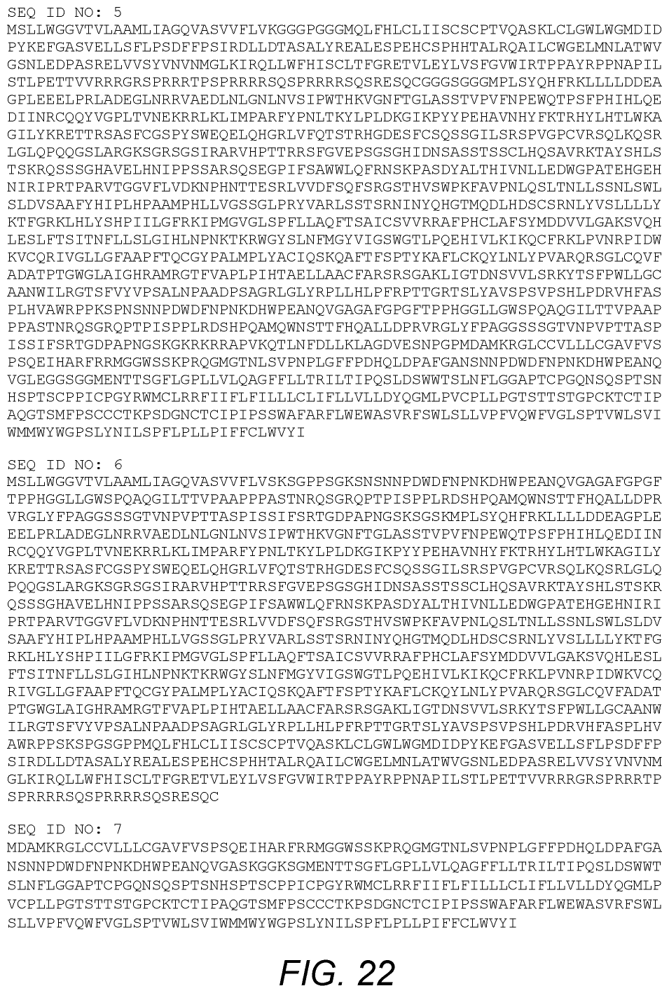

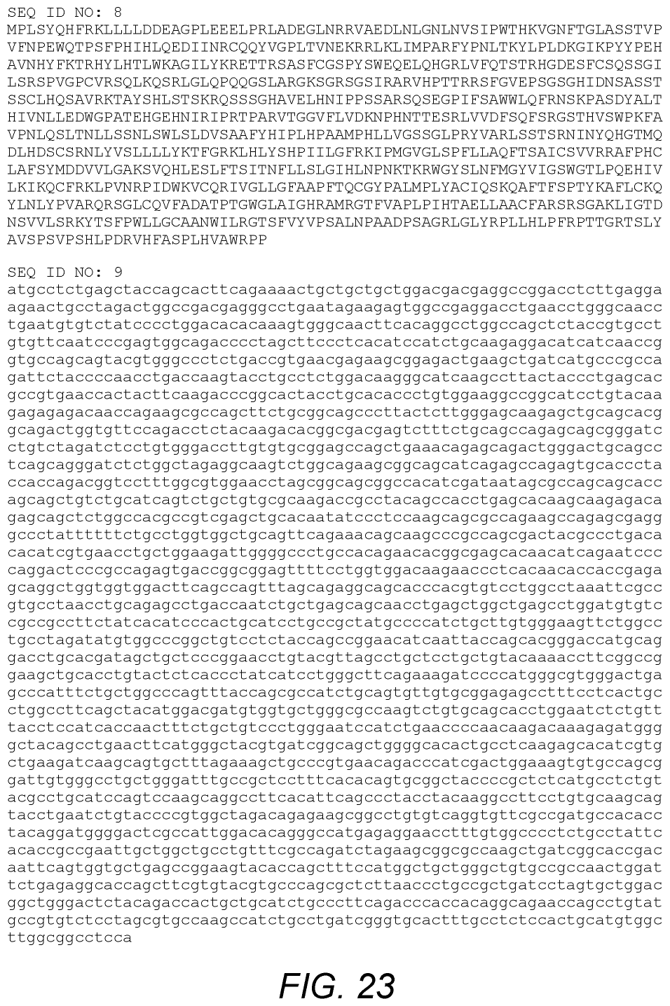

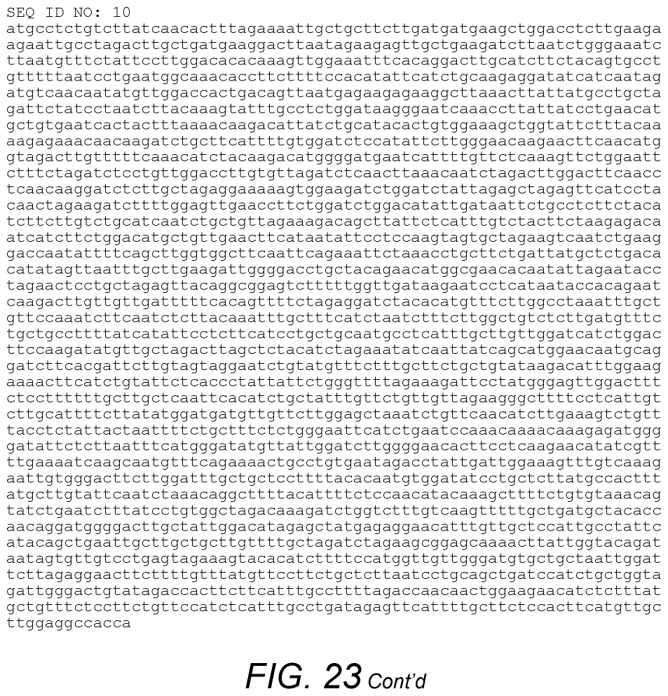

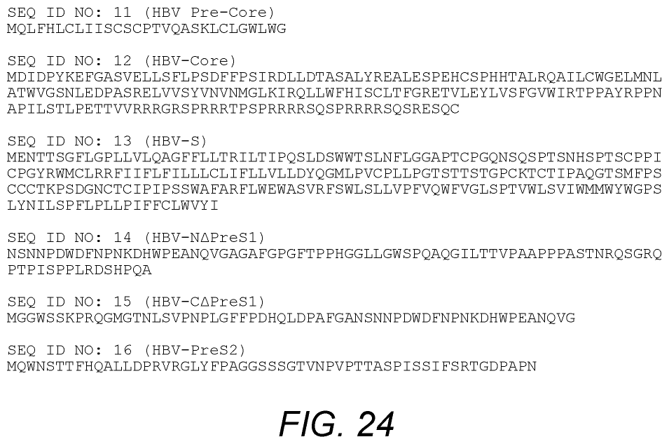

Response to core is shown in dark grey, to Pol1/2 in light grey, to Pol3/4 in white and to Pre-S1-2+S in black. FIG. 21 provides the same data as FIG. 20, plotted on the same scale as the CD8 IFNγ data for ease of comparison.

A robust T cell response was observed. The marked CD8+ T cell predominance has never been achieved by any other immunotherapeutic.

Tolerance of the Vaccine Regimen.

The vaccine regimen is safe and well tolerated. The heterologous prime-boost combination as monotherapy and in combination with LD nivolumab was safely administered, with no treatment-related SAEs, and infrequent transient transaminitis. At 26 Apr. 2022 no vaccine related SAEs had been documents. Two patients had mild rapidly resolving transaminitis. Local reactions have been mild or moderate. Details of patient reports are provided in Table 4. The table includes reactogenicity from both doses of the vaccine. Participants are included at most once per row.

TABLE 4

| Participants reporting | Any | Grade | Grade | Grade | Grade |
|---|---|---|---|---|---|
| (out of n = 43) | Grade | 1 | 2 | 3 | 4 |
| Any solicited symptom | 38 | 26 | 11 | 1 | 0 |
| Any local symptom | 35 | 27 | 8 | 0 | 0 |
| Pain | 33 | 26 | 7 | 0 | 0 |
| Redness | 2 | 2 | 0 | 0 | 0 |
| Swelling | 5 | 4 | 1 | 0 | 0 |
| Warmth | 16 | 13 | 3 | 0 | 0 |
| Any systemic symptom | 33 | 24 | 8 | 1 | 0 |
| Chills | 6 | 5 | 1 | 0 | 0 |
| Fatigue | 18 | 14 | 3 | 1 | 0 |
| Fever | 11 | 10 | 1 | 0 | 0 |
| Headache | 14 | 10 | 2 | 0 | 0 |
| Joint Ache | 16 | 10 | 6 | 0 | 0 |
| Malaise | 18 | 13 | 4 | 1 | 0 |
| Muscle Ache | 29 | 23 | 5 | 1 | 0 |
| Nausea | 8 | 6 | 2 | 0 | 0 |

### Example 5

Example 5 provides further data from the clinical trial described in Example 4 after more patients had progressed further into the treatment regimen, as detailed in Table 5.

TABLE 5

| | | | | | Through | |
|---|---|---|---|---|---|---|
| | | Gender | Total | Through | Mo | Through |
| Group | Age (Yrs) | (M:F) | participants | Mo 3 | 6 | Mo 9 |
| 1 | 52.6 +/− 7.3 | 8:1 | 10 (9)* | 9 | 9 | 9 |
| 2 | 53.3 +/− 6.9 | 15:3 | 18 | 18 | 12 | 8 |
| 3 | 49.8 +/− 8.5 | 11:7 | 18 | 18 | 10 | 7 |
| 4 | 49.9 +/− 9.7 | 7:2 | 9 | 9 | 9 | 7 |

*A 10^{th} participant has been omitted from the Group 1 summary due to a well controlled HBsAg (<LLoQ) at Baseline.

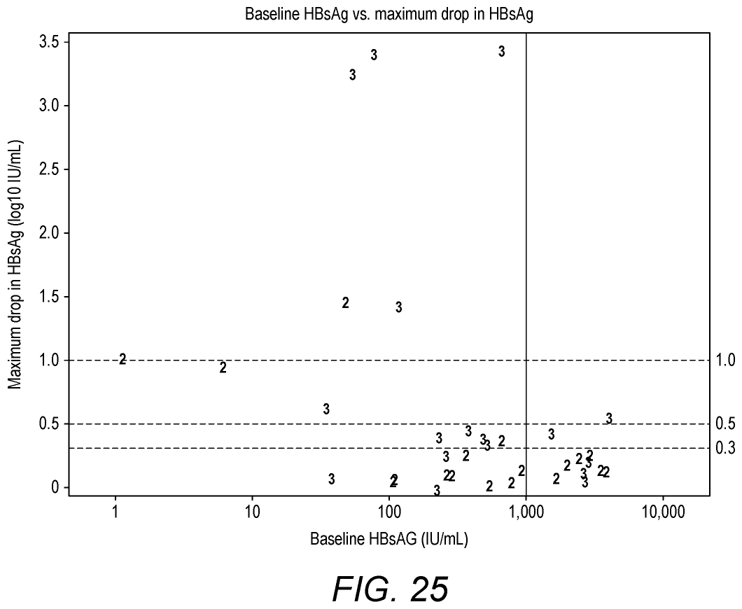

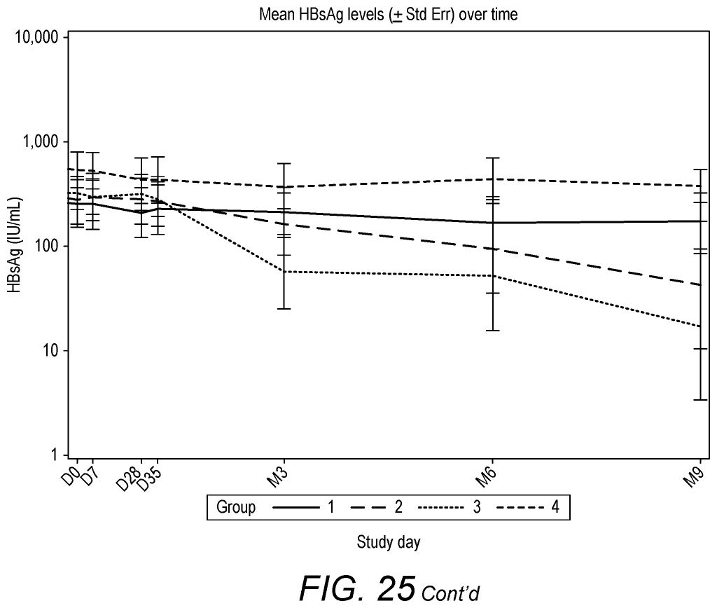

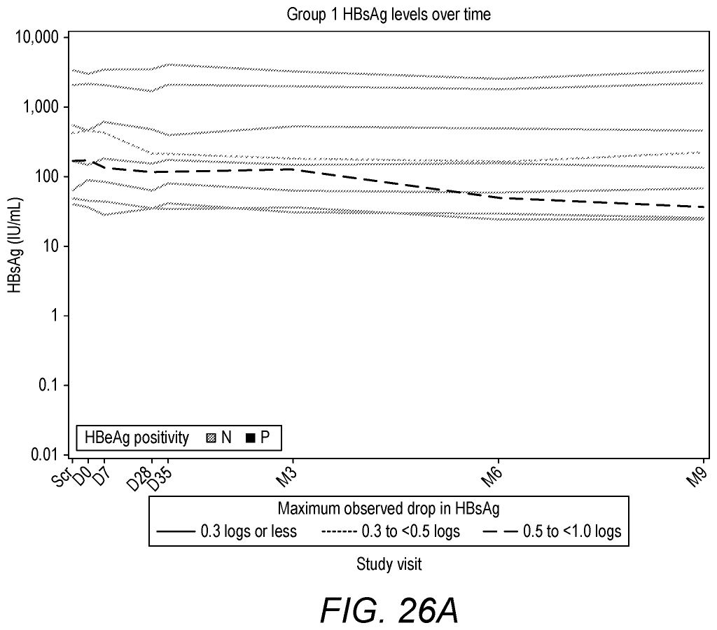

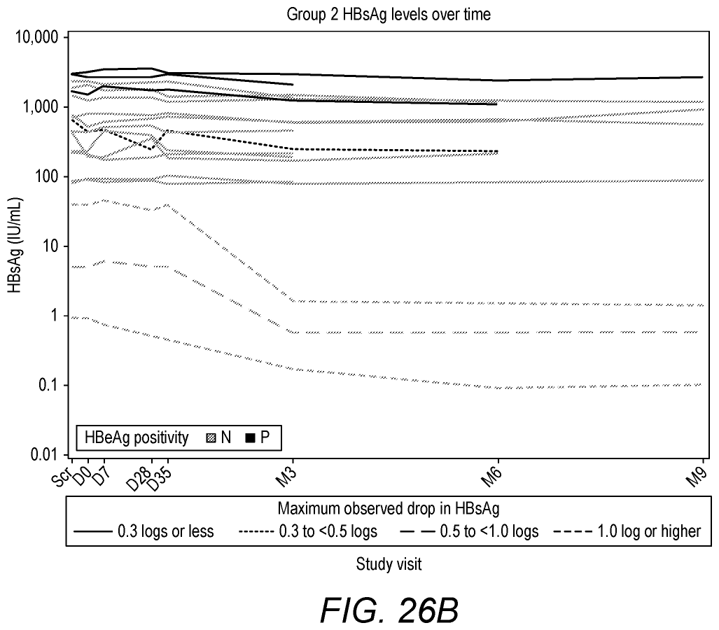

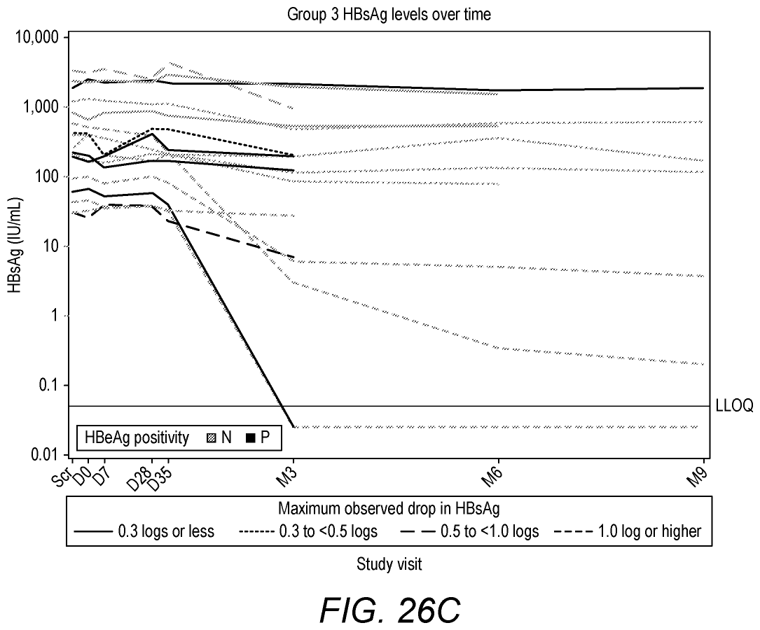

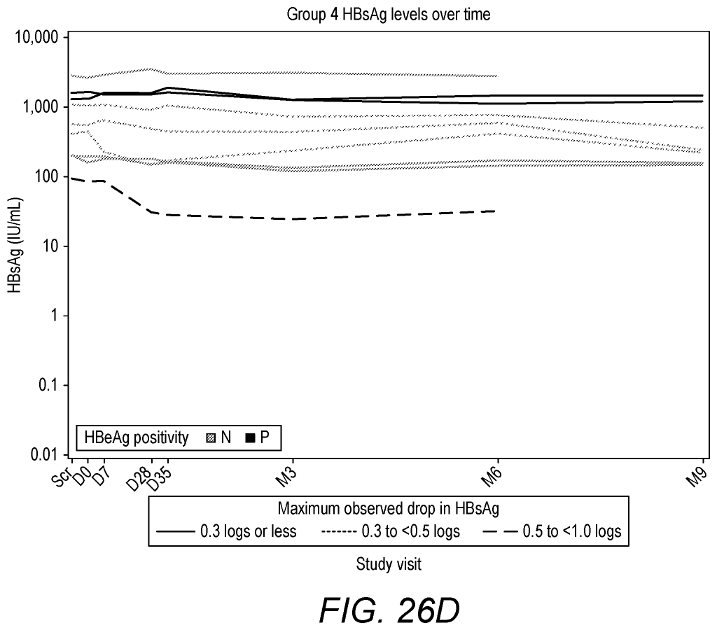

HBV-specific T cell responses were assessed using genotype C and D HBV peptides spanning the HBV immunogen in an IFNγ ELISpot assay, before and after (days 7, 28, 35, 84, and 168) administration as described in Example 4. All 55 patients were enrolled with no concerning safety signal or vaccine-associated SAE reported. Transaminase flares have been observed, associated with HBsAg decline, in two patients. Groups 1 and 4 had no appreciable change in HBsAg. In Group 2, three patients with starting HBsAg <50 IU/mL had declines of 0.9, 1.0, and 1.4 log₁₀ by Month 6 that persisted at the final timepoint of 9 months, i.e., 8 months after MVA-HBV administration. In Group 3, the mean log₁₀ reduction was 0.8 (N=18), 0.9 (N=10), and 1.3 (N=7) at Months 3, 6, and 9, respectively. FIG. 25 shows the HBV surface antigen responses by group, and FIG. 26 shows the surface antigen responses by individual. Individual plots show results for those with visits through months 3, 6 and 9.

HBV genotype C T cell responses were assessed in 20 patients to date, targeted HBV core (8/20), HBsAg (17/20) and pol (8/20). After prime vaccination, peak mean magnitude (day 7 or day 28) of total HBV specific T cell responses were 437, 244, 688, 332 SFU/10⁶ in Groups 1-4, respectively. After boost vaccination peak (day 35) total HBV specific T cell responses were 344, 689, 689, 277 SFU/10⁶ in groups 1-4, respectively. Responses were sustained out to 3-6 months in the majority of patients who received VTP-300 immunotherapy, either alone or combined with nivolumab at the boosting time point. Responses have also been immunogenic and show a reduction in HBsAg in well-controlled CHB patients, while exhibiting a well-tolerated safety profile.

Significant, durable reductions of HBsAg were seen in patients in the VTP-300 monotherapy group (Group 2). 3 of 5 patients with HBsAg below 100 IU/mL at baseline had 0.9, 1.0 and 1.4 log₁₀ declines 5 months post last dose, that persisted in all 3 patients at last follow-up 8 months post last dose. For patients who received VTP-300 with a single low dose of nivolumab at the time of the Day 28 booster dose (Group 3), the mean log₁₀ reduction in HBsAg was 0.8 (n=18), 0.9 (n=10), and 1.3 (n=7) at Months 3, 6, and 9, respectively. The effect was most prominent with starting values of HBsAg <1,000 IU/mL. 2 of 5 patients with HBsAg <100 IU/mL at baseline developed non-detectable HBsAg level at Month 3, which, in one patient with Month 6 and 9 visits, remained non-detectable (see FIG. 26). Genotyping of the HBV for individual patients shows that one of those two patients who developed non-detectable HBsAg levels at 3 months was infected with HBV serotype B and one patient was infected with HBV serotype C. 4 of 5 patients with HBsAg <100 IU/mL at baseline had declines >0.6 log₁₀. The lowering of HBsAg persisted in all patients with >0.5 log₁₀ reduction. No meaningful reductions were seen in Group 1 patients, who received 2 doses of MVA-HBV, or in patients who received low-dose nivolumab with both doses of VTP-300 (Group 4). These groups were discontinued after interim analysis. A robust T cell response against all encoded antigens was observed following VTP-300 administration, notable for marked CD8+ T cell predominance.

### Example 6

In Vivo Study to Assess T Cell Responses when a Checkpoint Inhibitor, Anti-PD-1, is Administered in a Heterologous Prime-Boost Regimen Using Viral Vectors Expressing Antigens from the Hepatitis B Virus (HBV)

A mouse study was performed to assess the T cell response when checkpoint inhibitor (α-PD-1) is co-administered with the prime or with the boost immunization, or as a stand-alone agent between prime and boost immunisations, in a heterologous prime-boost regimen using replication-incompetent viral vectors expressing antigens from the Hepatitis B virus (HBV). α-PD-1 is a well-characterised monoclonal antibody which acts as an immune modulator/checkpoint inhibitor. The particular clone used in this study is commercially available and supplied for in vivo use (InVivo Mab anti-mouse PD-1 (CD279) clone RMP1-14).

Female C57BL/6 mice were randomly allocated to experimental groups and allowed to acclimatise for a week. Vectors ChAdOx1-HBV, MVA-HBV and α-PD-1 were administered according to the schedule in Table 6, dosing at Day 0/Day 14/Day 28, by intramuscular (i.m.; ChAdOx1-HBV and MVA-HBV) or intra-peritoneal (i.p.; α-PD-1) injection. Spleens were harvested on Day 42.

Spleens were prepared into single cell suspensions for immunogenicity assessment in IFN-γ ELISpot assay. Splenocytes (200,000 per well) were restimulated for 18 hours with 1 μg/peptide/mL Pol 2 and Pre-S1/S2 peptide pools, in duplicate.

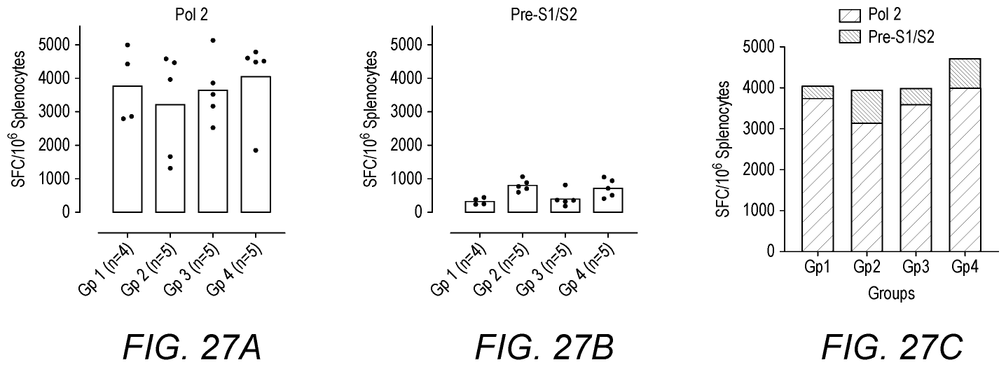

The results of the study are shown in FIG. 27. Panel A and B show the average SFC/10⁶ splenocytes generated in response to Pol 2 (panel A) and Pre-S1/S2 (panel B) by group. Each dot represents an individual mouse response. Panel C shows the group average SFC/10⁶ splenocytes. Stacked bars represent the average SFC/10⁶ splenocytes to both Pol 2 (pale grey) and Pre-S1/S2 (dark grey) peptide pools, by groups.

TABLE 6
Mouse study design

| | Day 0: | | Day 28: | Day 42: |
|---|---|---|---|---|
| Group | Prime | Day 14 | Boost | Harvest |
| 1 (n = 5*) | ChAdOx1-HBV | n/a | MVA-HBV | Spleens |
| 2 (n = 5) | ChAdOx1-HBV + | n/a | MVA-HBV | harvested |
| | α-PD-1 | | | on day 42 |
| 3 (n = 5) | ChAdOx1-HBV | n/a | MVA-HBV + | |
| | | | α-PD-1 | |
| 4 (n = 5) | ChAdOx1-HBV | α-PD-1 | MVA-HBV + | |
| | | | α-PD-1 | |

*For animal 1.1 in Group 1 the results were null including the control; sample excluded from analysis.

While preferred embodiments of the present invention have been shown and described herein, it will be obvious to those skilled in the art that such embodiments are provided by way of example only. Numerous variations, changes and substitutions will now occur to those skilled in the art without departing from the invention. It should be understood that various alternatives to the embodiments describes herein may be employed. It is intended that the following claims define the scope of protection and that methods and structures within the scope of these claims and their equivalents be covered thereby.

All documents referred to in this application are hereby incorporated by reference in their entirety.

### REFERENCES

Chinnakannan et al (2020): Design and Development of a Multi-HBV Antigen Encoded in Chimpanzee Adenoviral and Modified Vaccinia Ankara Viral Vectors; A Novel Therapeutic Vaccine Strategy against HBV. Chinnakannan et al. Vaccines. 2020 Apr. 14; 8(2).
Moore et al (2005) J. Immunology Vol 125, pages 7264-7273).

1. A method of vaccination, comprising administering to a subject a vaccine prime composition, a vaccine boost composition and a checkpoint inhibitor, the method comprising administering the vaccine boost composition and the checkpoint inhibitor at least 7 days after administration of the vaccine prime composition.

2. (canceled)

3. (canceled)

4. (canceled)

5. The method of claim 1, wherein the subject suffers from chronic hepatitis B virus (HBV) infection or cancer.

6. The method of claim 1, wherein the checkpoint inhibitor is a PD-1 inhibitor or anti-PD-1 antibody.

7. The method of claim 1, wherein the vaccine boost composition comprises a nucleic acid sequence encoding a target antigen is derived from HBV.

8. The method of claim 1, wherein the vaccine boost composition comprises a viral vectored vaccine.

9. The method of claim 8, wherein the viral vectored vaccine is a poxvirus.

10. The method of claim 1, wherein the vaccine boost composition comprises an RNA vectored vaccine.

11. The method of claim 1, wherein the vaccine boost composition and the checkpoint inhibitor are each administered separately or on the same day up to 56 days after the vaccine prime composition.

12. The method of claim 1, wherein the vaccine boost composition and the checkpoint inhibitor are administered on the same day, preferably wherein the vaccine boost composition and the checkpoint inhibitor are administered about 28 days after the vaccine prime composition.

13. The method of claim 1, wherein the checkpoint inhibitor is administered before the vaccine boost composition, wherein the checkpoint inhibitor is administered about 7 days after the vaccine prime composition and the vaccine boost composition is administered about 28 days after the vaccine prime composition.

14. The method of claim 1, wherein the vaccine prime composition comprises a viral vectored vaccine.

15. The method of claim 14, wherein the viral vectored vaccine is a simian adenovirus.

16. The method of claim 1, wherein the vaccine prime composition comprises a nucleic acid sequence encoding a target antigen derived from HBV.

17. The method of claim 16, wherein the vaccine prime composition comprises a nucleic acid sequence encoding the same target antigen as the vaccine boost composition.

18. The method of claim 1, wherein the vaccine prime composition and the vaccine boost composition comprise nucleic acid sequences encoding one or more antigens derived from HBV.

19. (canceled)

20. The method of claim 19, wherein the subject suffers from chronic HBV infection and has undergone antiviral therapy prior to administering the vaccine prime composition.

21. (canceled)

22. (canceled)

23. A kit comprising a vaccine prime composition, a vaccine boost composition and a checkpoint inhibitor as a combined preparation for separate, simultaneous or sequential use in a method of treatment of a viral infection or cancer.

24. (canceled)

25. The kit of claim 23, wherein the checkpoint inhibitor comprises an anti-PD-1 antibody, the vaccine prime composition comprises viral vectored vaccine ChAdOx1 and the vaccine boost composition comprises viral vectored vaccine Modified Vaccinia Virus Ankara (MVA).

26. The method of claim 1, wherein the checkpoint inhibitor comprises a PD-1 inhibitor or an anti-PD-1 antibody, the vaccine prime composition comprises viral vectored vaccine ChAdOx1, and the vaccine boost composition comprises viral vectored vaccine MVA.

27. The method of claim 26, wherein the subject suffers from an HBV infection or cancer.
